(19) United States
(12) Patent Application Publication
(10) Pub. No.: US 2010/0298403 A1
(43) Pub. Date: Nov. 25, 2010

(54) MODIFIED POLY(PROPYLENE-IMINE) DENDRIMERS AND THEIR USE AS TRANSFECTION AGENTS FOR AMIONIC BIOACTIVE FACTORS ( as amended

(75) Inventors: Frederik Tack, Dessel (BE); Henricus Marie Janssen, Eindhoven (NL); Egbert Willem Meijer, Waalre (NL); Michel Marie Francois Janicot, Mol (BE); Marcus Eli Brewster, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA N.V., Beerse (BE)

(21) Appl. No.: 11/994,576

(22) PCT Filed: Jul. 5, 2006
(86) PCT No.: PCT/EP2006/063888

§ 371 Date: Jan. 3, 2008

(30) Foreign Application Priority Data
Jul. 8, 2005 (EP) 05106266.9

(51) Int. Cl. A61K 31/7088; C08F 220/58; A61P 35/00; A61P 35/02

(52) U.S. Cl. 514/44 A; 526/310; 514/44 R

(57) ABSTRACT

The present invention is concerned with modified poly-(propylene imine) dendrimers, comprising cationic internal ammonium groups and external non-toxic endgroups, pharmaceutical compositions comprising said dendrimers, methods for the production of said dendrimers and their use as transfections agents for anionic bioactive therapeutic factors, for use in gene therapy, in particular for the treatment of cancer. The modified poly-(propylene imine) dendrimer of generation 1, 2, 3, 4 or 5, also comprising incomplete dendrimers and mixtures thereof, comprising external end groups and internal amine functions are characterized in that:
(a) substantially all external endgroups are groups of formula (I)

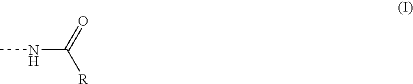

wherein R is a radical selected from the group of C₁₋₁₀alkyl, polyethylene glycol radical and polyethylene glycol gallyl radical; and
(b) substantially all internal amine functions are quaternary cationic ammonium functions. Most preferred are the quaternized compounds DAB-dendr-(NHCOCH₃)₄, DAB-dendr-(NHCOCH₃)₈, DAB-dendr-(NHCOCH₃)₁₆, DAB-dendr-(NHCOCH₃)₃₂, DAB-dendr-(NHCOCH₃)₆₄, DAB-dendr-(NHCOPh((EO)₄OMe)₃)₄, DAB-dendr-(NHCOPh((EO)₄OMe)₃)₈, DAB-dendr-(NHCOPh((EO)₄OMe)₃)₁₆, DAB-dendr-(NHCOPh((EO)₄OMe)₃)₃₂ and DAB-dendr-(NHCOPh((EO)₄OMe)₃)₆₄.

### FIELD OF THE INVENTION

The present invention is concerned with modified poly-(propylene imine) dendrimers, comprising internal cationic amine (ammonium) groups and external non-toxic end groups, pharmaceutical compositions comprising said dendrimers, methods for the production of said dendrimers and their use as transfections agents for anionic bioactive therapeutic factors, for use in gene therapy, in particular for the treatment of cancers.

### BACKGROUND OF THE INVENTION

Dendrimers are synthetic macromolecules with a well-defined, highly branched molecular structure that are synthesized in an algorithmic step-by-step fashion. Every repeated sequence of reactions produces a so-called ‘higher generation’ (G) molecule that has a practically doubled molecular weight and a doubled (discrete) number of functional end-groups. Since 1985, numerous chemically different types of dendrimers have been developed, such as Tomalia's poly(amido amino) PAMAM-dendrimers, Newkome's arborols, Fréchet's poly ether dendrimers, Meijer and Mülhaupt's poly(propylene imine) PPI-dendrimers and Moore's phenylacetylene dendrimers (Schlüter DA, 1999). Because of their defined structure, narrow polydispersity, defined nanoscale size and the ease of modification of the end groups, dendrimers are considered interesting candidates for various functions in life sciences and medicinal chemistry. In particular, their function as binding-and-release agents in drug- and as delivery vehicle in gene therapy has been investigated (Patri A K et al 2002; Esfand R et al. 2001, Liu M et al 1999, Stiriba S E et al. 2002, Bosman A W et al 1999, Tang M X et al 1997). Gene therapy is defined as the transfer of nucleic acids (such as DNA) into cells, preferably eucaryotic cells (such as human cells) to achieve a therapeutic effect. This effect can result from either correcting genetic defects or (over)expressing proteins that are therapeutically useful.

Among dendrimers, PAMAM dendrimers have received most attention as potential transfection agents for gene delivery, as these dendrimers are positively charged and can bind DNA at physiological pH. Some other dendrimer types have also been studied (Loup C et al. 1999, Choi J S et al. 2000, Ohasaki M et al. 2002, Shah D S et al. 2000, Liu M J et al. 1999, Joester D et al. 2003). Szoka et al. were the first to present DNA-transfection that was successfully mediated by PAMAM dendrimers, as evidenced by in vitro tests (Haensler J et al. 1993). Later, other studies on the association and transfection behavior of PAMAM-dendrimers have been published (Kukowska-Latallo J et al. 1996, DeLong R et al. 1997, Bielinska A et al. 1996, Shchepinov M S et al. 1997, Qin L et al. 1998, Yoo H et al. 1999, Cheng H et al. 2000, Ottaviani M F et al 2000, Kihara F et al. 2003). In particular, it has been found that heat-treated, partially degraded PAMAM-dendrimers perform better as in vitro DNA-carriers (Tang M X et al. 1996): These activated PAMAMs are commercially available under the name SuperFect (Qiagen©). Successful transfection for PAMAM dendrimers has been reported for charge ratios of around 5-20 (the charge ratio is defined as the number of terminal cationic amine sites in the PAMAM to the number of phosphates in DNA), i.e. an excess of transfection agent has to be used (Haensler J et al. 1993, Bielinska A U et al. 1999). PAMAM dendrimers of which a fraction of the terminal amines has been modified with glycol chains have also been introduced as potential DNA-transfection agents (Luo D et al. 2002). In this study, however, high concentrations of dendrimer have been used in the DNA-binding and transfection tests, while the use of agents that function at lower concentrations is in demand. Recent work on PAMAM dendrimers has given fundamental information on their interaction with cell-membranes (Hong S et al. 2004), also the next step in gene carrier design has been taken as PAMAMs with targeting antibody moieties have been prepared and studied (Thomas T P et al. 2004), and it has been shown that PAMAMs can interact with RNA-molecules resulting in inhibition of the activity of certain ribozymes (Wu J et al. 2005).

Important factors that determine the usefulness of a transfection agent are the toxicity and the efficiency of the agent. While some (Roberts J C et al. 1996) have shown that PAMAM dendrimers have toxicities depending on their generation and others (Szoka F C et al. 1996) have demonstrated that PAMAM dendrimers are less toxic than poly lysine (pLys), other data suggest that especially amine terminated PAMAM dendrimers show haemolitic and cytotoxic behaviour, whereas PAMAM dendrimers with terminal carboxylate groups are non-toxic (Duncan R et al. 1996, Malik N et al. 2000). Unfortunately, it seems that transfection is more efficient when PAMAM-dendrimers with high grades of amine-functionalization are used, presumably because this creates more cationic sites for DNA-binding at physiological pH (see for example FIG. 7 in Tang M X 1996).

Poly-(propylene imine) dendrimers are a specific class of dendrimers that have been developed at DSM Research© (Geleen, the Netherlands) (de Brabander-van-den Berg E M M et al. 1993) and independently in Mülhaupt's group (Wörner et al. 1993).

### Table 1 Shows the Molecular Characteristics of the Five Amine Terminated PPI-Dendrimers

| | | | external | internal |
|---|---|---|---|---|
| dendrimer | molecular | molecular | amine end | tertiary |
| generation | formula | weight | groups | amines |
| G1 | C₁₆N₆H₄₀ | 316.5 | 4 | 2 |
| G2 | C₄₀N₁₄H₉₆ | 773.3 | 8 | 6 |
| G3 | C₈₈N₃₀H₂₀₈ | 1686.8 | 16 | 14 |
| G4 | C₁₈₄N₆₂H₄₃₂ | 3513.9 | 32 | 30 |
| G5 | C₃₇₆N₁₂₆H₈₈₀ | 7168.1 | 64 | 62 |

They are commercially available at SyMO-Chem© (www.symo-chem.nl, Eindhoven, the Netherlands) and that can be worked with as starting materials for modification purposes. As an example, the molecular structure of the second generation PPI-dendrimer is represented in FIG. 1. PPI-dendrimers are characterized by their molecular weight, their external amine end groups and internal tertiary amine groups (see Table 1). Of course, due to incomplete reactions in the synthesis of each generation, dendrimers may be incomplete, and hence some internal amine functions may be secondary amine functions as well. In the context of this invention, it is understood that PPI-dendrimers refers to dendrimers of generation 1, 2, 3, 4 or 5, further comprising incomplete dendrimers and mixtures thereof, comprising a substantial number of internal tertiary amine groups before modification.

PPI-dendrimers with amine end groups degrade slowly in water and, more importantly, are too toxic to allow for their use in DNA-delivery systems, although reports on binding (Kabanov V A et al. 2000) and transfection (Zinselmayer B H et al. 2002) measurements have appeared. Data from literature strongly suggest that the terminal or surface groups (the exterior) of dendrimers determine the toxicity of the total dendritic structure, irrespective of the internal structure (Malik N et al. 2000). As a consequence, the surface of PPI-dendrimers can be modified chemically to create delivery systems with a low toxicity; additionally, surface modification can also promote water solubility and stability towards hydrolysis.

Apart from modification of the exterior, it is also possible to modify the interior of PPI or PAMAM dendrimers by quaternizing the internal tertiary amines to create cationic ammonium sites. In fact, quaternization of PPI-dendrimers has been reported before (Elissen-Roman C et al. 1997, Pan Y et al. 1999, Pan Y et al. 2000). Ford et al. (Kreider J L et al. 2001) have presented G2 and G4 PPI-dendrimers with short glycol chains at the exterior and quaternized interior sites, but the authors have not investigated or reported on their use as transfection agents. Recently, PAMAMs with cationically modified interiors have been reported as well: their transfection efficiency as measured with a luciferase gene expression test was lower than that of PEI or an unmodified PAMAM reference (Lee J H et al. 2003). Although the authors do not mention this, internally quaternized PAMAMs are most likely prone to exhibit retro Michael reactions, implying that these cationically modified dendrimers most likely degrade and are not stable.

### DESCRIPTION OF THE INVENTION

According to the present invention, a modified poly-(propylene imine) dendrimer is presented, wherein the poly-(propylene imine) dendrimer is modified at both the exterior and the interior with the aim to create water soluble, hydrolytically stable and non-toxic transfection agents for anionic bioactive factors. The PPI-dendrimers have been modified at the exterior by turning the amine end groups into groups of Formula (I)

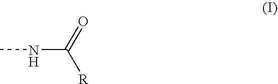

wherein R is a radical selected from the group of C₁₋₁₀alkyl, polyethylene glycol radical and polyethylene glycol gallyl radical, as these end groups preserve the water solubility, while it is proved that blocking the amine end groups generates non-toxic species.

The interior of the PPI-dendrimers has been modified by reacting the internal (predominantly tertiary) amine groups with a quaternization agent, such as methyl iodide, methyl chloride and the like, thus creating a micro-environment with multiple quaternary cationic sites. Depending on the generation of the PPI-dendrimer, the amount of cationic sites can be varied from 2 to 60 for the 1^{st} and 5^{th} generation, respectively, provided that the quaternization reaction proceeds quantitatively. The high local concentration of cationic sites in the interior of the dendrimer is anticipated to make this type of dendritic molecule well-capable of forming complexes with anionic bioactive factors.

Hence the invention relates to a modified poly-(propylene imine) dendrimer of generation 1, 2, 3, 4 or 5, also comprising incomplete dendrimers and mixtures thereof, comprising external end groups and internal amine groups, characterized in that:
a) substantially all external end groups are groups of formula (I), wherein R is a radical selected from the group of C₁₋₁₀alkyl, polyethylene glycol radical of formula

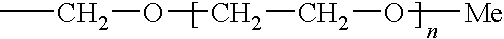

wherein n is 3, 4, 5, 6, 7, 8, 9, 10, 11, or 12; and
polyethylene glycol gallyl radical of formula

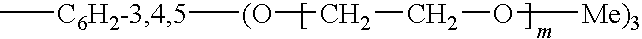

wherein each m independently is 1, 2, 3, 4, 5, 6, 7, 8, 9, 10, 11, or 12; and
(b) substantially all internal amine groups are cationic quaternary ammonium groups.

Furthermore, the invention relates to a modified poly-(propylene imine) dendrimer of generation 1, 2, 3, 4 or 5, also comprising incomplete dendrimers and mixtures thereof, characterized in that the modified poly-(propylene imine) dendrimer is obtained by:
(a) first reacting a poly-(propylene imine) dendrimer substantially comprising external amine end groups and internal tertiary amine groups, with an acylation agent selected from the group of acetic anhydride, a C₁₋₁₀alkyl halide, a polyethylene glycol acid of formula

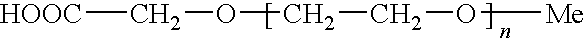

wherein n is 3, 4, 5, 6, 7, 8, 9, 10, 11, or 12; and
polyethylene glycol gallyl halide of formula

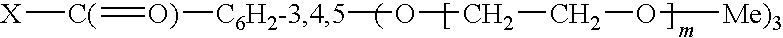

wherein each m independently is 1, 2, 3, 4, 5, 6, 7, 8, 9, 10, 11, or 12 and X is halide; and
(b) reacting the product obtained in step (a) with a quaternization agent.

Preferably, the C₁₋₁₀alkyl is methyl, ethyl, iso-propyl, n-propyl, t-butyl, n-butyl or pentyl. Most preferably, C₁₋₁₀alkyl is methyl.

As a halide, a chloride, bromide or iodide is preferred. A chloride is especially preferred.

Preferably, n is 3, 4, 5 or 6, most preferably 3 or 4.

Preferably, m is 3, 4, 5 or 6, most preferably 3 or 4.

As a quaternization agent, any agent that is known to the person skilled in the art to perform the desired task, i.e. converting a tertiary amine group into a quaternary ammonium group, may be used. Preferably, a methyl halide, most preferably methyl iodide is used, but also an agent comprising a C₁₀-alkyl group may also be used as a phase transfer agent.

With binding is meant any interaction that reversibly couples a chemical entity with at least one anionic site to at least one cationic site.

The invention is also directed to a pharmaceutical composition, suitable for administration to a mammal, preferably a human, characterized in that it comprises: (a) the modified poly-(propylene imine) dendrimer according to the invention; and (b) an anionic bioactive therapeutic factor.

As anionic bioactive factor is meant any chemical entity which is capable to bind to a cationic site, in particular pharmaceutical active compounds, nucleic acids, nucleic acid sequences, oligomers of DNA and RNA, polynucleotides, DNAzymes, single and double stranded DNA, single and double stranded RNA, antisense RNA and DNA, hammerhead RNA, short interfering RNA, micro RNA, ribozymes and the like; or combinations thereof.

Especially preferred are those anionic bioactive factors with a relatively low molecular weight, preferably equal to or less than 5,000 dalton, more in particular with a relative low number of base-pairs (oligo-DNAs or oligo RNAs, for example), preferably less than 50 base pairs. In this application, the inventors have used a 33-mer single stranded catalytic DNAzyme as a nucleic acid model to investigate the binding and transfection ability of the newly presented modified PPI-dendrimer. The transfection tests have been executed in vitro as well as in vivo.

Because of their low toxicity and their stability in serum and blood, the dendrimeric compounds of the present invention are suitable as transfection agents, and the pharmaceutical compositions comprising said compounds are especially suitable for use in gene therapy, most preferably in humans, more in particular for the treatment of cancer.

More preferably, the cancer is a tumor, associated with the liver, kidney, acute lymphoblastic leukemia, acute myeloid leukemia, Ewing's sarcoma, gestational trophoblastic carcinoma, Hodgkin's disease, non-Hodgkin's lymphoma, Burkitt's lymphoma diffuse large cell lymphoma, follicular mixed lymphoma, lymphoblastic lymphoma, rhabdomyosarcoma, testicular carcinoma, Wilms's tumor, anal carcinoma, bladder carcinoma breast carcinoma, chronic lymphocytic leukemia, chronic myelogenous leukemia, hairy cell leukemia, head and neck carcinoma, lung (small cell) carcinoma, multiple myeloma, follicular lymphoma, ovarian carcinoma, brain tumors (astrocytoma), cervical carcinoma, colorectal carcinoma, hepatocellular carcinoma, Karposi's sarcoma, lung (non-small-cell) carcinoma, melanoma, pancreatic carcinoma, prostate carcinoma, soft tissue sarcoma, breast carcinoma, colorectal carcinoma (stage III), osteogenic sarcoma, ovarian carcinoma (stage III), or combinations thereof.

The invention is also directed to a modified poly-(propylene imine) dendrimer of generation 1, 2, 3, 4 or 5, also comprising incomplete dendrimers and mixtures thereof, comprising external end groups and internal amine groups, characterized in that substantially all external end groups are groups of formula (I), wherein R is a radical selected from the group of C₁₋₁₀alkyl, polyethylene glycol radical of formula

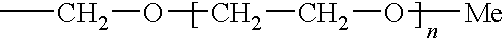

wherein n is 3, 4, 5, 6, 7, 8, 9, 10, 11, or 12; and polyethylene glycol gallyl radical of formula

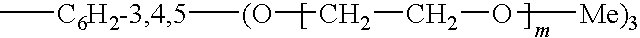

wherein each m independently is 1, 2, 3, 4, 5, 6, 7, 8, 9, 10, 11, or 12.

Furthermore, the invention relates to the a modified poly-(propylene imine) dendrimer of generation 1, 2, 3, 4 or 5, also comprising incomplete dendrimers and mixtures thereof, characterized in that the modified poly-(propylene imine) dendrimer is obtained by first reacting a poly-(propylene imine) dendrimer substantially comprising external amine end groups and internal tertiary amine groups, with an acylation agent selected from the group of acetic anhydride, a C₁₋₁₀alkyl halide, a polyethylene glycol acid of formula

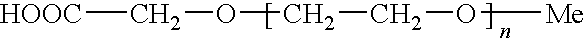

wherein n is 3, 4, 5, 6, 7, 8, 9, 10, 11, or 12; and
polyethylene glycol gallyl halide of formula

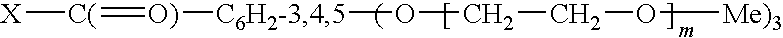

wherein each m independently is 1, 2, 3, 4, 5, 6, 7, 8, 9, 10, 11, or 12 and X is halide.

The invention will now be elucidated and explained in more detail with a number of experiments, without being delimited thereto.

### EXPERIMENTAL

### 1. Syntheses of Modified Poly-(Propylene Imine) Dendrimers

### 1.1. General

The synthesis of poly-(propylene imine) dendrimers modified with glycol gallate groups has been described in literature (see Baars, M. W. P. L., Kleppinger, R., Koch, M. H. J., Yeu, S. L., Meijer, E. W., Angew. Chem. Int. Ed. Engl., 2000, 39, 1285 and the supporting information to this article). For the synthesis of glycol gallate (i.e. gallic acid or 3,4,5-trihydroxybenzoic acid, decorated with three monomethoxy tetraethylene glycol groups) the same reference can be consulted. Quaternized polypropylene imine) dendrimers with acetyl or poly ethylene glycol gallate groups, have not been reported in literature before.

Poly(propylene imine) dendrimers with amine end groups are available from SyMO-Chem (www.symo-chem.nl) and are usually denoted as DAB-Am-4 (generation 1), DAB-Am-8 (generation 2), DAB-Am-16 (generation 3), DAB-Am-32 (generation 4) and DAB-Am-64 (generation 5), for the first, second, third, fourth and fifth generation, respectively. DAB stands for the 1,4-diaminobutane core, Am stands for the amine end groups and the given number stands for the number of end groups.

Applied solvents routinely are of p.a. quality. Used solvents and reagents include methyl alcohol (Biosolve p.a.), toluene (Biosolve p.a.), dichloromethane (Biosolve p.a.), water (demineralized over column), triethylamine (Fluka, >99%, stored on KOH-pellets), acetic anhydride (Acros p.a.), oxalylchloride (Acros) and methyl iodide (Merck, stored in refrigerator).

Dowex 1×8-50 (Acros) Cl⁻-anion exchange resin with a capacity >1.2 meq/ml (Acros) and Dowex 550A OH (25-35 mesh) strongly basic OH⁻ anion exchange resin (Aldrich) have been used. The success of the ion exchange from iodide to chloride can be checked by performing a test. First, a few mg of the dendrimer product is dissolved in about 1 mL water and some drops of concentrated H₂O₂ (35%-solution, Merck) are added. At this stage the I⁻ containing dendrimer solution colors somewhat yellow, while the Cl⁻-containing dendrimer solution remains colorless (the slight coloration is due to the formation of I₂). After addition of about 1 mL of freshly prepared starch solution, the I⁻-containing dendrimer solution becomes dark blue, whereas the Cl⁻-containing dendrimer induces no coloration of the solution. The starch solution is obtained by adding soluble starch powder (1 g, Merck) to well-stirred boiling water (100 mL). After one minute, the solution is allowed to cool down and used immediately for the test.

TABLE II
Modified poly(propylene imine) dendrimers.

| | | molec- |
|---|---|---|
| | corresponding | ular |
| formula dendrimers | short formula | weight* |
| DAB-dendr-(NHCOCH₃)₈ | G2 | 1109 |
| DAB-dendr-(NHCOCH₃)₈ + 6 MeI | G2 MeI | 1961 |
| DAB-dendr-(NHCOCH₃)₈ + 6 MeCl | G2 MeCl | 1413 |
| DAB-dendr-(NHCOCH₃)₃₂ | G4 | 4859 |
| DAB-dendr-(NHCOCH₃)₃₂ + 30 MeI | G4 MeI | 9117 |
| DAB-dendr-(NHCOCH₃)₃₂ + 30 MeCl | G4 MeCl | 6374 |
| DAB-dendr-(NHCOPh((EO)₄OMe)₃)₃₂ | G4 PEG | 26644 |
| DAB-dendr-(NHCOPh((EO)₄OMe)₃)₃₂ + | G4 PEG MeI | 30903 |
| 30 MeI | | |
| DAB-dendr-(NHCOPh((EO)₄OMe)₃)₃₂ + | G4 PEG MeCl | 28161 |
| 30 MeCl | | |
| DAB-dendr-(NHCOPh((EO)₄OMe)₃)₆₄ | G5 PEG | 53429 |
| DAB-dendr-(NHCOPh((EO)₄OMe)₃)₆₄ + | G5 PEG MeI | 62228 |
| 62 MeI | | |

Table II lists the modified poly(propylene imine) dendrimers that have been synthesized.

As dialysis membranes, Spectrum Laboratories Spectra/Por tubes were used (various cutoff MWCO materials have been applied). Reactions were routinely executed under an inert atmosphere of argon. NMR analyses have been performed on a Varian Mercury Vx 400 MHz or a Varian Gemini 300 MHz spectrometer. After isolation the prepared dendrimers were routinely stored at −20° C. or at 4° C. in the dark.

### 1.2. DAB-dendr-(NHCOCH₃)₈ or “G2”

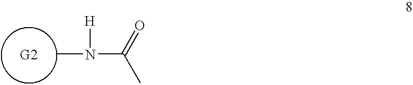

The second generation amine terminated poly-(propylene imine) dendrimer (6.50 g; 8.40 mmol; FW=773) was dissolved in methyl alcohol (50 mL) and triethylamine (6.8 g; 67.24 mmol). Acetic anhydride (8.24 g; 80.8 mmol) was added during 1 minute (reflux; no external cooling). After stirring for 2.5 hours, the solution was evaporated on a rotavap and stripped once with methyl alcohol. A column was charged with Dowex 550A OH (25-35 mesh), and the ion exchange resin was washed with water and then with methyl alcohol (this is somewhat exothermic). The crude dendrimer in methanol was eluted in a drop-wise fashion in order to give the exchange process enough time. The product was isolated by rotary evaporation and stripping with methanol followed by vacuum evacuation using an oil pump. A clear colorless oil was acquired.

¹H NMR (CD₃OD): δ=8.1 (t), 3.2 (t), 2.5 (m), 1.9 (s), 1.7 (m), 1.5 (m). ¹³C NMR (CD₃OD): δ=173.1, 55.2, 53.3, 53.2, 52.7, 39.0, 27.7, 25.8, 24.9, 22.7. ES/MS M⁺=1109.4.

### 1.3. DAB-dendr-(NHCOCH₃)₈+6 MeI or “G2(MeI)”

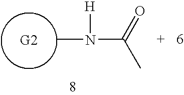

### Ideally all 6 Internal Tertiary Amines are

The acylated second generation polypropylene imine) dendrimer (725 mg) was dissolved in methyl alcohol (2 mL) and methyliodide (4.6 g). The solution was stirred at an oil bath temperature of 50° C. for 20 hours under an argon atmosphere. After evaporation of the volatiles a yellowish brittle powder was obtained.

¹H NMR (CD₃OD): δ=8.0 (t), 3.9 (b), 3.7-3.5 (b), 3.3 (m), 2.5 (b), 2.2 (b), 2.05 (b), 2.0 (s). ¹³C NMR (CD₃OD): δ=173.5, 62.8, 61.8, 60.2, 59.8, 50.0, 37.3, 23.9, 23.3, 20.7, 19.3.

### 1.4. DAB-dendr-(NHCOCH₃)₈+6 MeCl or “G2(MeCl)”

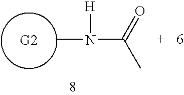

### Ideally all 6 Internal Tertiary Amines are

The acylated and methyliodide quaternized second generation poly-(propylene imine) dendrimer (309 mg) was dissolved in methyl alcohol (2 mL) and applied to a column charged with Dowex 19×8-50 ion exchange resin that had been washed with water and methanol. Elution was executed with methyl alcohol. Evaporation of the filtrate resulted in the MeCl-adduct (0.21 g).

¹H NMR (CD₃OD): δ=3.7 (b), 3.6-3.4 (b), 3.3 (m), 2.4 (b), 2.0 (b), 1.95 (s).

### 1.5. DAB-dendr-(NHCOCH₃)₃₂ or “G4”

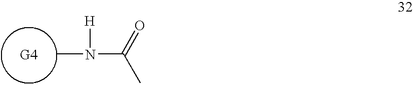

The fourth generation amine terminated poly-(propylene imine) dendrimer (2.02 g; 0.57 mmol; FW=3514 g/mol) was dissolved in dichloromethane (50 mL) and triethylamine (2.05 g; 20.3 mmol). Acetic anhydride (2.15 g; 21.06 mmol) was added dropwise during one minute (exothermic reaction, no external cooling). After overnight stirring, methyl alcohol (20 mL) was added resulting in a clear solution that was stirred for another 3 hours. The solution was evaporated and stripped three times with methyl alcohol. A methanol solution of the product was eluted on a pre-washed column of Dowex 550A OH (25-35 mesh) ion exchange resin. The eluate was evaporated on a rotavap, stripped with methanol repeatedly and dried in vacuo resulting in a viscous oil (2.7 g).

¹H NMR (CD₃OD): δ=3.2 (t), 2.5 (m), 1.9 (s), 1.7 (m), 1.5 (m). ¹³C NMR (CD₃OD): δ=173.0, 53.5-52.7, 39.0, 27.8, 25.0-24.5, 22.5.

### 1.6. DAB-dendr-(NHCOCH₃)₃₂+30 MeI or “G4(MeI)”

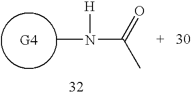

### Ideally all 30 Internal Amines are

The acylated fourth generation poly-(propylene imine) dendrimer (FW=4859 g/mol; 1.0 g; 0.206 mmol; 6.17 mmol internal tertiary amines) was dissolved in methyl alcohol (2 mL) and methyl iodide (7 mL). The mixture was stirred for 60 hours at an oil bath temperature of 45° C. The volatiles of the two-layer mixture were evaporated giving a yellow powder. This product was dissolved in methyl alcohol and precipitated in well-stirred ether. A finely divided yellow powder was obtained.

¹H NMR (CD₃OD): δ=8.2 (b), 4.1-3.5 (b), 3.3 (m), 2.8-2.5 (b), 2.3-2.1 (b), 2.05 (s). ¹³C NMR (CD₃OD): δ=173.5, 61.8, 60.4, 59.7, 51.2, 50.2, 37.5, 23.9, 23.5, 20.5, 19.4.

### 1.7. DAB-dendr-(NHCOCH₃)₃₂+30 MeCl or “G4(MeCl)”

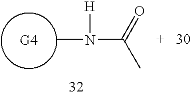

### Ideally all 30 Internal Amines are

The acylated and methyliodide quaternized fourth generation poly-(propylene imine) dendrimer was dissolved in methyl alcohol and applied to a column charged with Dowex 1×8-50 ion exchange resin that had been washed with water and methanol. Elution was executed with methyl alcohol. Evaporation of the filtrate resulted in the MeCl-adduct.

¹H NMR (CD₃OD): δ=8.3 (b), 3.9-3.2 (b), 2.7-2.4 (b), 2.1-2.0 (b), 2.0 (s). ¹³C NMR (CD₃OD): δ=173.5, 61.5, 60.4, 60.0, 59.6, 49.9, 37.4, 23.6, 22.9, 20.7, 18.4.

### 1.8. Glycol Gallyl Chloride Building Block, Cl(O)C-Ph((EO)₄OMe)₃

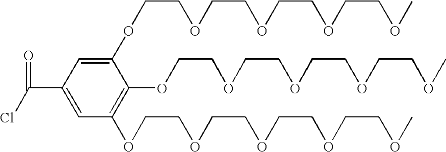

Glycol gallate (HOOC-Ph((EO)₄OMe)₃)(2.05 g, 2.68 mmol, FW=741) was stored over powdered P₂O₅ in vacuum. Before use, it was stripped (co-evaporated) twice with toluene. Then, it was dissolved in 60 mL of distilled dichloromethane and 2.8 mL of oxalyl chloride was added, followed by 3 drops of DMF. An extra portion of 0.2 mL oxalyl chloride was added after an hour, as IR analysis still showed a peak at 1714 cm⁻¹ (COOH-group). An additional 10 minutes stirring gave complete conversion to the acid chloride (IR: 1745 cm⁻¹). The product Cl(O)C-Ph((EO)₄OMe)₃ was isolated by evaporation of the solvents on a rotary evaporator and co-evaporation with toluene. It was immediately used in coupling reactions with poly-(propylene imine) dendrimers.

### 1.9. DAB-dendr-(NHCOPh((EO)₄OMe)₃)₃₂ or “G4-PEG”.

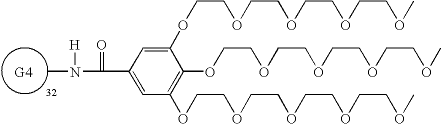

The fourth generation amine terminated poly-(propylene imine) dendrimer (276 mg; FW=3514 g/mol), was stripped four times with toluene and was dissolved in 6 mL dichloromethane and 1 mL triethylamine. This solution was added within half a minute to a solution of the acid chloride Cl(O)C-Ph((EO)₄OMe)₃ in 60 mL of dichloromethane (1.1 eq. of acid chloride was used). A clear solution resulted. After overnight stirring, 30 mL water and 550 mg KOH powder was added, and the whole mixture was transferred to a separation funnel. The organic layer was separated, and the water layer was extracted with 50 mL dichloromethane. The combined dichloromethane layers were washed with a solution of 200 mg KOH in 25 mL water and then with two 25 mL portions of water. The dichloromethane solution was dried with anhydrous sodium sulfate, filtered and concentrated to yield 1.85 g of oily product. This product was dialyzed twice with methanol/water/triethylamine 500:60:10 (v/v/v) and finally with methanol/water 500:25 (v/v). After evaporation, co-evaporation with methanol to remove the last triethylamine and drying in a vacuum stove, a slightly yellowish oily product was acquired (1.24 g).

¹H NMR (CDCl₃): δ=8.0 (bs), 7.1 (bs), 4.1 (b), 4.0 (b), 3.8-3.4, 3.35 (s), 3.3 (s), 2.5-2.2 (b), 2.0-1.4 (b). ¹³C NMR (CDCl₃): δ=167.1, 152.3, 141.0, 129.7, 106.8, 72.4, 72.1, 70.8, 70.7, 69.8, 69.0, 58.9, 53-51 (b), 38.6, 27.0, 24.0.

### 1.10. DAB-dendr-(NHCOPh((EO)₄OMe)₃)₃₂+MeI or “G4-PEG(MeI)”

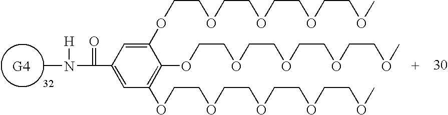

### Ideally all 30 Internal Tertiary Amines are

The fourth generation poly-(propylene imine) dendrimer modified with glycol gallate groups (590 mg) was stirred for 40 hours in 5 mL methanol and 2 mL methyliodide at 40-45° C. (oil bath temperature) in a round bottomed flask equipped with reflux condenser. The solution was evaporated down on a rotary evaporator, and the product was subsequently stripped three times with methanol. Yield: 0.69 g of a viscous yellow-brown oil.

¹H NMR (CD₃OD): δ=7.2 (bs), 4.2 (b), 4.0-3.4, 3.35 (s), 3.3 (s), 2.8-2.4 (b), 2.3-2.1 (b).

### 1.11. DAB-dendr-(NHCOPh((EO)₄OMe)₃)₃₂+MeCl or “G4-PEG(Mea)”

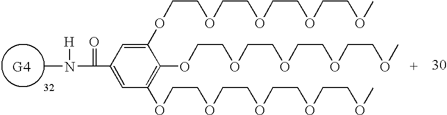

### Ideally all 30 Internal Tertiary Amines are

A column with 3.0 g of Dowex 1×8-50 (Acros) ion exchange resin was washed with demineralized water and with methanol to remove contaminants. The G4-dendrimer with glycol gallate groups quaternized with MeI (the I⁻-form; 290 mg) was dissolved in 5 mL methanol and put on the column. Elution with methanol was continued until fractions did not show any UV-activity on a silica-60 TLC plate. The methanol solution was evaporated on a rotavap to yield 247 mg of product (viscous slightly yellow oil).

¹H NMR (CDCl₃): δ=8.5 (b), 7.3 (bs), 4.3-4.1 (b), 3.9-3.4, 3.35 (s), 3.3 (s), 2.8-2.4 (b), 2.3-2.0 (b). ¹³C NMR (CDCl₃): δ=167.5, 152.3, 140.8, 129.0, 106.8, 72.3, 71.8, 70.5, 69.6, 69.0, 60.4 (b), 58.9, 49.5-49.0 (b), 37.1 (b), 22.5 (b), 17.2 (b).

### 1.12. DAB-dendr-(NHCOPh((EO)₄OMe)₃)₆₄ or “G5-PEG”

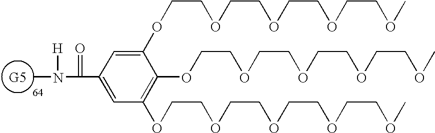

The fifth generation amine terminated poly-(propylene imine) dendrimer (251 mg containing ca. 20 wt % methanol) was stripped three times with toluene to remove the methanol and was then dissolved in a mixture of dichloromethane (40 mL) and triethylamine (250 mg). Freshly prepared acid chloride (Cl(O)C-Ph((EO)₄OMe)₃; 1.2 eq. per primary amine) in 20 mL was added during one minute to the vigorously stirred dendrimer solution. The solution became turbid immediately. After overnight stirring under an argon atmosphere, the mixture was concentrated on a rotavap. The product was dissolved in water (5 mL) and a sodium hydroxide solution (300 mg in 5 mL water). Purification was achieved by dialysis versus MeOH/water/triethylamine (400/40/40 mL) and then versus MeOH/water (500/50 mL). Evaporation and drying (oilpump) resulted in 1.44 g of oily product.

¹H NMR (CDCl₃): δ=8.2 (b), 7.1 (bs), 4.1 (b), 3.9 (b), 3.8-3.4, 3.35 (s), 3.3 (s), 2.5-2.3 (b), 1.8-1.3 (b). ¹³C NMR (CDCl₃): δ=167.1, 152.3, 141.0, 129.7, 106.8, 73-68, 59, 53-51, 38.9, 27.0, 24.0.

### 1.12. DAB-dendr-(NHCOPh((EO)₄OMe)₃)₆₄+62 MeI or “G5-PEG(MeI)”

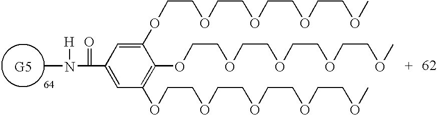

### Ideally all 62 Internal Tertiary Amines are

The fifth generation poly-(propylene imine) dendrimer modified with glycol gallate groups (150 mg) was dissolved in 2 mL methanol. A solution of methyliodide (360 mg) in methylalcohol (0.5 mL) was added and the resulting mixture was stirred for 20 hours at 40° C. (temperature of oil bath) under an inert argon atmosphere. The volatiles were evaporated on a rotavap resulting in a yellow-brownish product.

¹H NMR (CD₃OD): δ=7.3 (bs), 4.3 (b), 3.8-3.4, 3.35 (s), 3.3 (s), 2.8-2.4 (b), 2.3-2.1 (b). ¹³C NMR (CD₃OD): δ=168.9, 153.7, 142.2, 130.3, 107.9, 73.7-71.3, 61.8-60.0 (b), 59.2, 50.7 (b), 38.5 (b), 24.2 (b), 19.5 (b).

### 2. Stability Measurements Using ¹H NMR, ¹³C NMR and SEC in Aqueous Mixtures

The dendrimers were dissolved in D₂O, and the solutions were transferred to NMR-tubes. The tubes were placed in an oil bath that was kept between 35 and 39° C. during four days. Every day the ¹H NMR spectrum was recorded, before and after the four days the ¹³C NMR spectra were taken. The pH-values were also recorded during the measurement: DAB-dendr-(NHCOCH₃)₈ remained basic (pH=ca. 9), DAB-dendr-(NHCOCH₃)₃₂+30 MeI remained acidic (pH=ca. 2) and DAB-dendr-(NHCOCH₃)₈+MeCl remained slightly acidic during the measurement (pH=ca. 5-6), whereas DAB-dendr-(NHCOCH₃)₈+MeI developed from slightly acidic (pH=5-6) to being more acidic (pH=ca. 2). The acidity of the solutions containing the methyliodide quaternized dendrimers is probably caused by the presence of some HI that had formed during the quaternization procedure in MeOH/MeI. The pH of the solution was assessed every day by simply using universal pH-indicator paper. For the SEC-measurements a similar procedure was applied. A TSK-GEL G3000PWₓₗ column was used, applying a 0.5 mL/min flow of a 0.1 M citric acid and 0.05% sodium azide in water eluent; RI-detection was used.

### 3. Binding Experiments of Dendrimers and DNA Using Polyacrylamide Gel Electrophoresis (PAGE)

PAGE was executed using a BIO-RAD Mini-PROTEAN 3 Cell. Mini gels of 17% cross linking density were prepared by mixing 5.7 mL of a 30% acrylamide and 2.67% bis-acrylamide solution with 1.0 mL buffer solution (10×), 3.3 mL H₂O, and, directly before casting of the gel between the spaced glass plates, 60 μL of a freshly made 10% ammonium persulfate (APS) solution and 10 μL of TEMED. For the pH=4.4 gels, a double amount of TEMED and APS solution was applied. The solutions were allowed to gel for at least an hour, before running the gels. In all cases 18 MΩ water was used.

A Tris/Boric Acid/EDTA buffer (TBE buffer; 10×) containing 108 gram Tris (890 mM), 55 gram H₃BO₃ (890 mM) and 7.5 gram EDTA (20 mM) per liter was used in the experiments conducted at a pH of 7. For measurements at pH=4.4, a β-alanine/acetic acid buffer was employed containing 12 gram acetic acid (197 mM) and 71.2 gram of β-alanine (800 mM) per liter (10×). The loading buffer contained 0.2 mL of a 1% bromophenol blue solution in H₂O, 25 mL buffer (1×) and 15 mL glycerol. The loading sample consisted of appropriately chosen volumes of a DNA-solution in water, a dendrimer solution in water and the loading buffer solution. Every lane on the gel was loaded with 10 μL or 12.5 μL of loading sample, such that the DNA-load per lane was about 0.4 μg (unless noted otherwise), and such that the dendrimer/DNA charge ratio (CR) was about 2:1, 3:2, 1:1 or 1:2 for the various inspected dendrimer/DNA combinations. The charge ratios were calculated by dividing the amount of positive charges in the dendrimer (i.e. the total amount of tertiary and quaternary amines in the dendrimers) by the amount of phosphate groups in the DNA. The employed DNA was a single stranded unlabeled 33-mer. On every gel, as references, one lane was reserved for the unlabeled ss-DNA and one lane for a mixture of this ss-DNA 33-mer with a FITC-labeled ss-DNA 33-mer. Some lanes were not used.

The mini gels were run for about 45 minutes at a voltage of 200 Volts. Ag-staining with a standard BIORAD kit and a standard BIORAD protocol was used to develop the gels. In all cases, white lines on a slightly brown background were obtained; the contrast and brightness of all pictures of the gels were manipulated in such a way that black lines on a white or grayish background were obtained.

In FIG. A, one can assess the binding capacities of dendrimers G2(MeCl), G4(MeI), G4-PEG(MeCl), G5-PEG and G5-PEG(MeI) with the DNA-zyme 33-mer at pH=7.

In FIG. B, the concentration binding study at pH=7 is shown: DNA-loads increase from 0.1 to 0.2 to 0.4 to 0.8 microgram per lane (with a loading volume of 12.5 microliter), using the indicated charge ratio of dendrimer G4-PEG(MeI).

### 4. Cells, Animals and Materials

The following, all human, cell lines were used in this study: the mammary carcinoma MCF7 cell line and the malignant melanoma Malme-3M cell line, both cultured in dulbecco's minimum essential medium. The ovarian carcinoma A2780 cell line, the colorectal adenocarcinoma cell line HT29 and the leukemia cell line K562-C1000 were cultured in RPMI 1640. These culture media were supplemented with 5% fetal calf serum (FCS), 50 μg/ml gentamycin, and 2 mM L-glutamine. MCF7 cell culture medium and Malme-3M culture media was also supplemented with 1 mM sodium pyruvate. Cells were grown at 37° C. in a humidified incubator with 5% CO₂. All media and supplements were purchased from Invitrogen (Paisley, UK).

Male NMRI mice were purchased from Janvier (Le Genest-St-Isle, France). All animal experiments were carried out with animal ethical committee approval. The ethical guidelines that were followed met the standards required by the UKCCCR guidelines.

5′-Fluorescein-labeled and non-labeled ss-DNAzyme 33-mers (5′-label-TGAGGGGCAGGCTAGCTACAACGACGTCGCGGx-3′) with x=3′ dG5′ were purchased from Eurogentec (Seraing, Belgium). In order to improve stability, a 3′-3′ guanine inversion was incorporated at the 3′-end.

### 5. Cellular Toxicity of Modified PPI-Dendrimers

The toxicity of modified PPI-dendrimers of different generations (G2, G2(MeI) and G2(MeCl), G4, G4(MeI) and G4(MeCl) and G5-PEG and G5-PEG(MeI)) was profiled on 4 cell lines (Malme-3M, K562, HT29 and MCF7) using the MTT-test. A cytotoxicity assay, in which cells were plated at 2000 cells/well in 96-well plates 24 hours prior to transfection, was used for this purpose. The dendrimer was added to the cells at various concentrations dependent on the generation of dendrimer. The 2nd generation dendrimer was added at concentrations ranging from 500 μM to 1 μM. The 4th and the 5th generation dendrimers were added at concentrations ranging from 100 μM to 0.2 μM and from 12.5 μM to 50 nM, respectively. Cells were treated with the dendrimer for 4 hours and then refreshed with complete media and further incubated for 4 days. After this incubation period the cells were checked for the mitochondrial dehydrogenase enzyme, which is only present in living cells. When present, an added yellow MTT salt will be reduced by the enzyme to form a blue formazan crystal, which can be dissolved in DMSO and measured using a spectrophotometer (λmax at 540 nm). The found absorption is then divided by the absorption of cells that undergo the same experimental procedure, but that are untreated with the dendrimer, to give the MTT-viability versus the control that is displayed in all Figures.

An MTT protocol was also followed when the cytotoxicity of a range of modified 4th generation PPI-dendrimers was examined in more detail. Acetylated or pegylated dendrimers, either quarternized (with MeI or MeCl) or non-quarternized were tested, employing a concentration range (from 1 μM, 2 μM, 5 μM, 10 μM to 20 μM), while also investigating an increasing amount of serum (10%, 20%, 30%, 40%). These MTT-tests were performed on the A2780 cells that were chosen due to their suitability as in vivo models in our department.

### 6. In Vitro Delivery of Fluorescently Tagged DNAzymes

A fluorescence activated cell sorter (FACS) analysis was performed to determine the cellular uptake of the FITC-labeled DNAzyme using 4th generation dendrimers as transfecting agents. 2*10E+6 A2780 cells/well were seeded in a 6 well plate 24 h prior to transfection. The dendrimer and DNAzyme were both diluted in culture medium to a final concentration of 1 μM, achieving a charge ratio of about 1 upon complexation. A 15 minute incubation period enabled complexation of the two components. The complex was subsequently added to the cells and after a 4 hour incubation, cells were washed twice with PBS, collected by trypsinization, washed twice in FACS buffer and Cell Scrub Buffer (Gene Therapy Systems, San Diego, Calif.). Propidium Iodide was added to each sample at a final concentration of 20 μg/ml to determine the quantity proportion of dead cells. Finally, the cells were analyzed for DNAzyme uptake by flow cytometry (FACScan, Becton Dickinson). Non-transfected cells were applied as baseline control to determine auto-fluorescence of the cell. Cells treated with DNAzyme alone were applied as negative control. Thus, auto-fluorescence and transfection due to the DNAzyme alone are accounted for in the values of the transfection efficiencies in all Figures.

### 7. In Vivo Delivery of Fluorescently Tagged DNAzymes

### 7.1. Microscopy

A whole body imaging (WBI) system was used to investigate the in vivo tumor delivery of fluorescently tagged DNAzymes. This imaging system consists of a fluorescence stereomicroscope (Olympus) SZX12 equipped with a green fluorescent protein (GFP) (excitation: 485-501 nm; emission: 510 nm) and a red fluorescent protein (RFP) (excitation 540-552 nm; emission: 568-643 nm) filter set (see for details: Bakker A, Floren W, Voeten J, Janssens B, Smets G, Wouters W and Janicot M (2001) Automation of whole body imaging of GFP-expressing tumors in living animals. G.I.T. Imaging and Microscopy March 2001:52-54). Images (752×582 pixels) were acquired at 1/60th of a second using a (Jai) CV-M90 3-CCD RGB color camera and analyzed using in-house developed application software that is based on IMAQ Vision software components and LabVIEW (National Instruments).

Intracellular DNAzyme delivery was investigated on tumor sections using fluorescence microscopy. Briefly, at the end of each animal experiment, fluorescent tumors were extracted, cryofixed and sectioned. 12 μm sections were observed using a AxioPlan2 (Zeiss) fluorescence microscope coupled to a AxioCam HR (Zeiss) CCD camera and high resolution pictures (1300×1030 pixels) were captured and further analyzed using AxioVision software (Zeiss). Intracellular distribution of FITC (green) labeled DNAzyme was investigated using a nuclear dye TOPRO3 (red). β-Actin staining was obtained using bodipy phalloidin (blue).

### 7.2. DNAzyme Administration In Vivo

Male NMRI mice were injected in the inguinal region with 107 A2780 ovarian carcinoma cells/200 μl serum-free medium using 26GA syringes (BD, 26 GA 3/8 1 ml). After 14 days the tumors had reached adequate size for WBI measuring. Of a first group of mice, the control mice (n=5) were treated Iv. with 1 mg FITC-conjugated c-myc DNAzyme (FITC-DNAzyme), while the test mice (n=5) were treated with dendrimer-DNAzyme complex formulations containing 1 mg of FITC-DNAzyme and ca. 3 mg of G4-PEG(MeI) achieving a CR of 1 (i.e. a final concentration of 50 μM of DNA and dendrimer in the mouse, if dilution into a blood volume of ca. 2 mL in the mouse is assumed). Intravenous (i.v.) injections were administered via the tail vein at injection rates of ˜200 μl/10 sec. The mice (n=10) were sacrificed at 45′ (minutes) and the tumor was immediately cryofixed in TissueTek (Triangle Biomedical Sciences). Apart from these treated mice (n=10), a few untreated mice were used as a negative control. A second group of mice (n=10) were injected with the same DNAzyme-dendrimer complex and were checked after a period of 24 h for fluorescence in the tumor using WBI. After 24 h this second group of mice was sacrificed and the interior was examined for fluorescence. DNAzyme clearance after iv. injection was monitored by WBI at 5′ (minutes), 15′, 30′ and 45′ (for the first group of mice) and at 24 hrs (for the second group of mice) post-injection.

### Results and Discussion

### 1. Synthesis and Characterization of Dendritic Transfection Agents for Nucleic Acids.

The synthesis of polypropylene imine) transfection agents is summarized in FIG. 2 illustrating the conversion steps of the 2^{nd} generation dendrimer. PPI-dendrimers of other generations have been converted in an analogous way. In the first step, the primary amine endgroup is amidated by reaction with an activated carboxylic acid derivative “RCOOH” (either acetic anhydride or a gallyl chloride derivative have been used here; other types of activated carboxylic acids are also possible, see e.g. Kreider J L et al. 2001). In the second step, the interior tertiary amines are quaternized by reaction with methyliodide. In the third step, the iodide counter-anion is exchanged for chloride.

All prepared dendrimers are soluble in water and alcohols and most of them are also soluble in more apolar solvents such as chloroform. The recorded ¹H NMR and ¹³C NMR data are in agreement with the assigned structures. In CDCl₃ and in CD₃OD, all protons and carbons from the dendrimer interior are quite broad, especially for the higher generation dendrimers. Upon methylation (quaternization) the broad signals around 2.2-2.5 ppm from the methylene protons adjacent to the tertiary amines shift to 3.5-4.0 ppm for the acetylated dendrimers and to around 2.7-2.9 ppm for the pegylated dendrimers. In ¹³C NMR one sees for both type of dendrimers that these methylene carbons shift from around 50-55 ppm to around 60 ppm, while extra signals appear at around 50 ppm for the introduced methyl groups. In D₂O the signals of the methylenes next to the tertiary or quaternary amines are less broad. All the NMR-data are in agreement with earlier reported results on other quaternized dendrimers.

It should be noted that the NMR data prove that the quaternization has proceeded quite well, but they do not prove that methylation has occurred completely, i.e. not necessarily all tertiary amines have been converted to quaternized cationic sites. It is possible to acquire mass data on the lower generation unquaternized dendrimers, but MS-analysis of the quaternized dendrimers has failed so far, possibly due to the many charges on the dendritic molecules. For convenience, all drawn structures in this paper are the perfect, totally methylated species (FIG. 2).

As a final characterization tool, it is possible to analyze the prepared dendrimers using size exclusion chromatography (SEC), a technique that is used to investigate the molecular weight (distribution) of macromolecules. Applying a TSK-GEL G3000PWₓₗ column and employing aqueous eluents at a pH of choice (e.g. a 0.1 M citric acid buffer at lower pH-values), all prepared PPI-dendrimers both unquaternized and quaternized can be analyzed. This technique has been applied to assess the stability of the prepared dendrimers (see the next section).

### 2. Stability of the Modified PPI-Dendrimers in Water

The designed and prepared dendrimers can only be useful as transfection agents if their stability under physiological conditions is ensured. Therefore, a selection of dendrimers has been tested by daily monitoring the ¹H NMR and ¹³C NMR spectra of D₂O-solutions of these dendrimers that were kept at ca. 37° C. during 4 days. Spectra have been recorded for the 2^{nd} generation dendrimers G2, G2(MeI) and G2(MeCl), and the 4^{th} generation dendrimer G4(MeI). All dendrimers show similar spectral characteristics before, during and after the test period of 4 days, so that significant hydrolysis of the dendrimers is not indicated under the mimicked physiological conditions.

Even stronger evidence for the stability of (quaternized) dendrimers has been acquired by monitoring aqueous solutions kept at 37° C. of 4^{th} generation dendrimers G4-PEG and G4-PEG(MeCl) applying SEC. All SEC data are illustrated in FIG. 3. During the several days of the experiment, the SEC-chromatograms of the samples do not change their shape at all, so no evidence for degradation, that should result in lower molecular weight material being formed, has been found. The SEC-trace shows a shoulder at the higher molecular weight (left) side of the chromatogram. Presumably, this shoulder points at the presence of limited amounts of dimerized dendrimer species. It should be noted that the shoulder is also present in the SEC of the fourth generation amine terminated dendrimer that is the starting point of the synthesis.

### 3. Dendrimer-DNAzyme Binding Experiments Using PAGE

Polyacrylamide gel electrophoresis (PAGE) is a technique that is frequently used in the analysis of proteins and nucleic acids. The elution of the species under investigation is dependent on its size and on its charge. SDS-PAGE (addition of sodium dodecyl sulfate to the gel-buffer), for instance, is applied to assess the molecular weight of (unfolded) proteins.

Here, PAGE has been performed to investigate the binding properties of several poly(propylene imine) dendrimer structures to a DNAzyme molecule (for details see the experimental section). After elution and staining the free DNAzyme appears as a single band on the gel. If upon mixing of the DNAzyme with another species binding occurs, the elution behavior of the DNAzyme in the mixture alters compared to that of the free DNAzyme, as the volume and/or the charge of the DNA-species has changed. This results in a DNA-band at the same position, but with a lower intensity (a fraction of the DNA is complexed), in a band at another position on the gel or in a complete disappearance of the band. To assess the binding capacity of the set of prepared dendrimers, the DNAzyme and the dendrimer have been mixed in different charge ratios, where the charge ratio is defined as the number of tertiary plus quaternary amines in the dendrimer divided by the number of negatively charged phosphate groups on the DNA.

The binding of the DNAzyme molecule with the different dendrimers given in Table 2 has been investigated. Here, three gels are selected and shown in FIG. 4 below to illustrate the findings; the gels were prepared and run in a TBE buffer of pH=7. In the Supplementary Information more of the recorded PAGE gels are collected.

Gel A shows a comparison between the acylated and methyliodide quaternized dendrimers G2(MeI) and G4(MeI). Clearly, the fourth generation dendrimer binds the DNA-zyme better than the second generation counterpart that does not seem to induce binding at the investigated concentration. This observation can be explained by the fact that the G4(MeI) dendrimer bears twice as many cationic sites per molecule (30 versus 14) and thus its design is more matched to the 33 negative charges in the DNA-zyme. Gel B compares acylated fourth generation dendrimers that are quaternized (G4(MeCl)) or not (G4). Both dendrimers are able to bind the DNA-zyme quite effectively, but the quaternized G4(MeCl) species is more potent, as even at a ½ dendrimer-DNA charge ratio almost all DNA is bound. Finally, gel C compares glycolgallate modified fourth generation dendrimers that are quaternized (G4-PEG(MeI)) or not (G4-PEG). Again it is clear that the quaternized species bind the DNA-zyme better, but the results also shows that acylated G4-dendrimers (see gel B) give a better binding to the DNA than the pegylated G4-dendrimers, as higher excesses of the pegylated dendrimers are necessary to bind the DNA-zyme effectively.

The binding properties of the dendrimers at a pH-value of 4.4 using an acetic acid/13-alanine buffer has also been studied (no gels shown). In comparison with the measurements at pH=7, the investigated unquaternized dendrimers seem to bind better, while the quaternized dendrimers bind the DNA to a somewhat lesser extent. This result can be explained by the protonation of the unquaternized dendrimers at lower pH-values, so that these dendrimers also have multiple cationic sites in their interior, promoting binding to the DNAzyme.

Finally, G4-PEG(MeI) has been selected for a concentration-range binding study. DNA-loads per lane of 0.1, 0.2, 0.4, 0.8 and 1.6 microgram in 12.5 microliter have been used, while charge ratios were varied from 2:1 to 3:2 to 1:1 (excess dendrimer). Naturally, the PAGE-study shows that binding is reduced at lower concentrations: at a load of 0.1 microgram the DNA is almost completely unbound, while at loads 0.8 microgram or higher all DNA is bound even at the lowest charge ratio of 1:1 (see the Supplementary Information for the acquired PAGE-gels of this concentration binding study).

The results demonstrate that the synthesized dendrimers with multiple cationic sites in their interior can bind the ss-DNAzyme 33-oligomer at concentrations of around 40 microgram DNA per mL (corresponding to a molar concentration of about 4 μM), and at charge ratios of around 2:1-1:1 (slight excess dendrimer). Transfection agents reported in literature usually require higher concentrations and/or higher charge ratios to allow for efficient complexation with the DNA guest (see for example Haensler J 1993 where some binding tests have been executed at DNA-concentrations of 200 μg/mL). Moreover, the concentration-range binding study on the G4-PEG(MeI) species shows that binding between dendrimer and DNAzyme is reversible, enabling dissociation of the complex and release of the DNAzyme.

### 5. In Vitro Toxicity of Modified PPI-Dendrimers

In order to assess their suitability as gene transfection agents, the toxicity of a selection of modified PPI-dendrimers has been investigated on 4 different cell lines, MCF7, Malme-3M, HT29 and K562-C1000, using the MTT-test. The 2nd generation acylated dendrimers G2, G2(MeI) and G2(MeCl) do not exert a toxicity on all 4 cell lines below concentrations of 100 μM, while the 4th generation acylated dendrimers G4, G4(MeI) and G4(MeCl) show no sign of toxicity below a level of 20 μM for these cells. Finally, the 5th generation dendrimers G5-PEG and G5-PEG(MeI) are non toxic at the highest level investigated (2.5 μM). These concentrations are 20, 20 and 5 times higher than the levels that were used for standard in vitro transfection experiments with the respective 2nd, 4th and 5th generation dendrimers.

The toxicity of 4th generation dendrimers has been investigated in particular, as the 4th generation dendrimers have been found to bind the DNAzyme more effectively than their 2nd generation counterparts (see the PAGE tests described above). For each of the six studied dendrimers (i.e. G4, G4(MeI), G4(MeCl), G4-PEG, G4-PEG(MeI) and G4-PEG(MeCl) the cellular toxicity was assessed using the MTT test, while applying varying dendrimer (1 μM, 2 μM, 5 μM, 10 μM and 20 μM) and serum concentrations (10%, 20%, 30% and 40% fetal calf serum). As can be seen in FIG. 5, at concentrations of 1-5 μM, all six dendrimers exert no specific toxicity and >70% of the cells survive after a 4 day treatment. At higher concentrations, however, especially G4-PEG(MeI) shows a definite toxicity as cell survival rates drop clearly below 50%. The other dendrimers still show a low cell death of about 30% at a concentrations of 10 μM, and a partial toxicity of 30-60% at 20 μM. G4-PEG(MeCl) retains its low toxicity even at a 20 μM level. FIG. 6 represents the same MTT-test data in an other fashion, categorized per dendrimer and indicating the increased toxicity at higher concentrations. A serum level of 10% was used in the data shown in FIG. 5 and FIG. 6.

Each of the six G4-dendrimers has also been tested for its toxicity in the presence of increasing quantities of serum, applying levels from 10% to 40%. All dendrimers exert a lower cellular toxicity when higher quantities of serum are used (FIG. 7). Remarkably, when 20%-40% of serum is used, the toxicity of the dendrimers seems (almost) independent of the concentration that is used; even at a 20 μM level, the cell survival is clearly above 50% for all dendrimers, except for dendrimer G4-PEG(MeI) that becomes toxic at concentrations above 10 μM.

It can be concluded from the MTT toxicity tests described here that almost all designed and prepared PPI-dendrimers show low levels of toxicity. This property of the compounds according to the invention is extremely important as a low or absent toxicity is a condition sine qua none for a successful use in humans, in particular in gene therapy. Possibly, the counter anions in the quaternized dendrimers determine to some extent the toxicity of the species, and it seems favorable to use chloride counter anions in stead of iodides.

### 6. In Vitro Transfection of DNAzyme Using Modified G4 PPI-Dendrimers

The transfection of DNAzyme using 4th generation modified PPI-dendrimers as delivery agents has been investigated on A2780 ovarian carcinoma cells applying a FACS analysis. A dendrimer-DNAzyme charge ratio of CR=1 and a concentration of 1 μM is used in all transfection tests to remain a low level of toxicity (see the MTT toxicity tests presented above), and to stay in the concentration domain where binding between DNA and dendrimer is anticipated (see the PAGE binding test presented above). An increasing level of serum in the medium has been examined (10%, 20%, 30% and 40% FCS) to mimic in vivo conditions.

All 6 dendrimers show high transfection efficiencies usually exceeding 80%, with the acetylated quaternized dendrimers G4(MeI) and G4(MeCl) displaying the best results (FIG. 8). Remarkably, in the class of the pegylated dendrimers (FIG. 8B), there is hardly a difference in efficiency when the unquaternized system G4-PEG is compared to the quaternized systems G4-PEG(MeI) and G4-PEG(MeCl). Finally, the transfection tests show that the amount of serum in the medium does hardly affect the efficiency of in vitro delivery. Free DNAzyme, i.e. no dendrimer transfection agent is used, transfects with an efficiency of only 5-10%, as established in a control experiment. In a confirmation of the toxicity tests, a cellular toxicity of approximately 15% has been found in these in vitro delivery tests, as revealed by propidium iodide staining

The transfection efficiencies found for the dendrimer systems are similar to what is found in the same setup when a cationic liposomal transfection agent, DOTAP® (Roche®), is used (MW=ca. 700). However, this liposome can only neutralize one negative charge per molecule, so that the amount needed to reach a CR of 1 is such that the toxicity of the transfection mix is high. Concludingly, the use of DOTAP delivery methods seems disabled in vivo.

### 7. In Vivo Delivery of DNAzyme Using Modified G4 PPI-Dendrimers

The preliminary in vivo experiments that are presented here have been executed using the G4-PEG(MeI) dendrimer. The acetylated quaternized dendrimers G4(MeI) and G4(MeCl) that show the most convincing in vitro transfection ability have been rejected for this purpose, as they produce insoluble precipitates when mixed with the DNAzyme at concentrations that are required for preparing samples for in vivo studies (for example, G4(MeI) and DNAzyme give a white precipitate when mixed at a concentration of ca. 700 μM, a concentration that is much higher than those used in the binding, toxicity or in vitro transfection tests described above).

After treating the mice intravenously with the dendrimer/FITC-labeled DNAzyme complex, the fluorescence has been visualized using whole body imaging (WBI). After 5 minutes the fluorescence is visible everywhere in the body. After 45′ the fluorescence is no longer externally detectable in three of the five mice using WBI, although the fluorescence had locally accumulated in the beginning of the duodenum as was visualized after dissection post mortem. Two of the five mice, however, show a weak externally visible fluorescence near the tumor. These two samples have been analyzed using confocal microscopy to determine whether the fluorescence observed with WBI is indeed intracellular co-localized. 24 h after injection no fluorescence can be seen, neither externally nor internally post mortem.

In the two samples that show externally visible accumulation of the FITC label, sectioning the tumor and analyzing the section via confocal microscopy results in an intensive spotty like accumulation of the FITC label in the tissue. The reason for this spotty like pattern and for the extensive accumulation in the nucleus is still unclear. There is a high co-localization with the TOPRO3 (red) dye indicating the FITC label is present in the nucleus. An additional staining (Bodipy phalloidin (blue)) has been performed to observe the β-actin levels of the cell.

In the sections prepared from the tumors excised from the treated mice large lumen like holes are seen and appear to be present in the vicinity of the majority of the accumulated FITC label. The sections obtained from the tumors from the untreated mice did not have these lumen like structures. Also this is still unclear as to how and why these lumen are present. Whether they work as a trap for the oligoDNA or if they created by the dendrimer-DNAzyme complex or a part of the complex (the dendrimer alone) once separated requires further investigation. Non treated samples have a much better β-actin staining around the nucleus. This is however almost not detectable in the areas that contain FITC labeling in the treated samples.

### CONCLUSION

As the field of post transcriptional gene silencing is constantly advancing with new additional players e.a. short interfering (si) RNA and micro (mi) RNA, the field of drug delivery is under more and more pressure to present a better and safer transfection agent to accommodate the needs in oligonucleotide therapy. Although most delivery agents on the market claim to achieve high transfection efficiency and low toxicity in a large number of cell types, at present none have profilated themselves as a decent in vivo drug delivery tool.

In this application, modified PPI-dendrimers have been described—some of which have never been reported before—that can easily be prepared and that can act as transfection agents in gene therapy. It was demonstrated that the designed and prepared PPI-dendrimers are stable in aqueous environments and that these dendrimers enable in vitro delivery of an ss-DNAzyme oligomer into ovarian carcinoma cells, while inducing only a low cellular toxicity. The delivery is efficient as the binding and transfection of the DNAzyme can proceed at low concentrations and low charge ratios (i.e. low excesses of dendrimer still enable transfection). Moreover, preliminary in vivo experiments show that delivery is feasible. Finally, initial PAGE binding studies have shown that double stranded siRNA (44 nucleotides) also bind to the PPI-dendrimers described here, so that binding and transfection of nucleic acids does not seem to be restricted to the ssDNAzyme model that has been used in this application as a model.

### LIST OF FIGURES

FIG. 1: Molecular structure of the second generation PPI-dendrimer.

FIG. A: Binding capacities of dendrimers G2(MeCl), G4(MeI), G4-PEG(MeCl), G5-PEG and G5-PEG(MeI) with the DNA-zyme 33-mer at pH=7.

FIG. B: Concentration binding study at pH=7

FIG. 2: The synthesis of poly(propylene imine) transfection agents, illustrating the conversion steps of the 2^{nd} generation dendrimer.

FIG. 3: Stability of the modified PPI-dendrimers in water (SEC data).

FIG. 4: Dendrimer-DNAzyme binding experiments using PAGE.

FIG. 5: In vitro toxicity of modified PPI-dendrimers: MTT-test data, categorized per charge ratio.

FIG. 6: In vitro toxicity of modified PPI-dendrimers: MTT-test data, categorized per dendrimer.

FIG. 7: In vitro toxicity of modified PPI-dendrimers under presence of increasing concentrations of serum: MTT-test data, categorized per charge ratio and per dendrimer

FIG. 8: In vitro transfection efficiency of modified PPI-dendrimers: FACS analysis, categorized per dendrimer

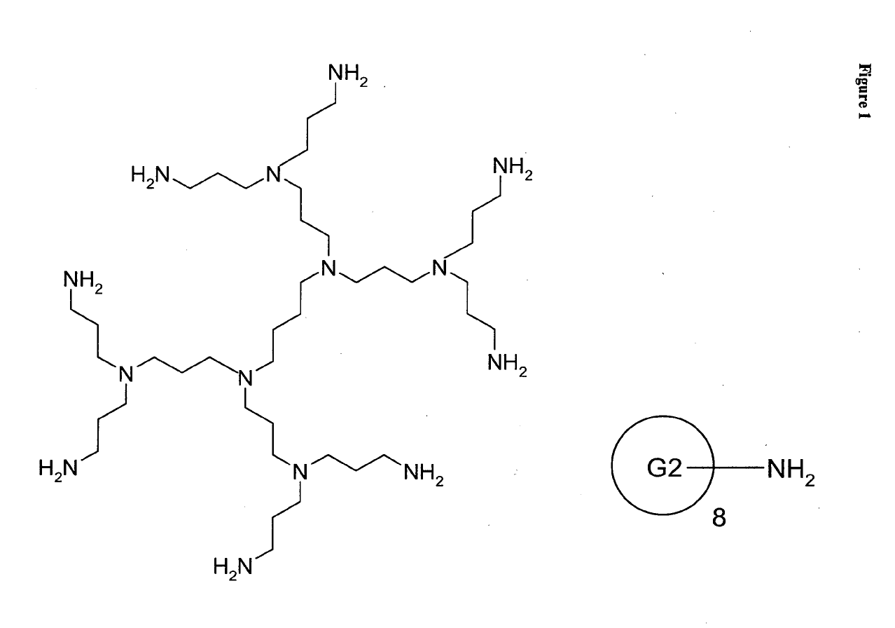

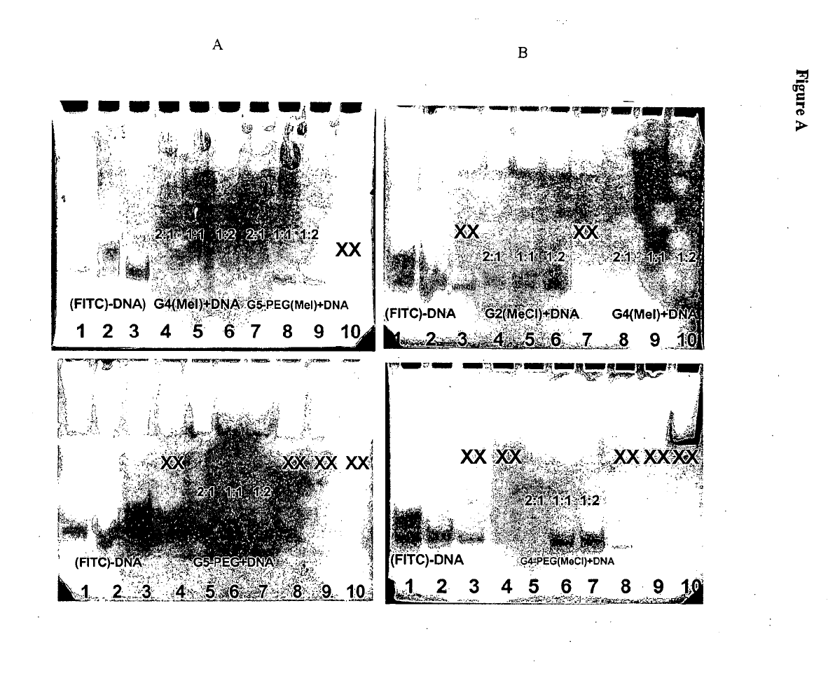

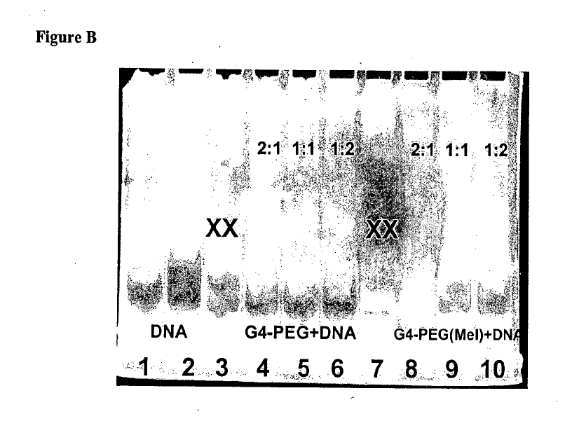

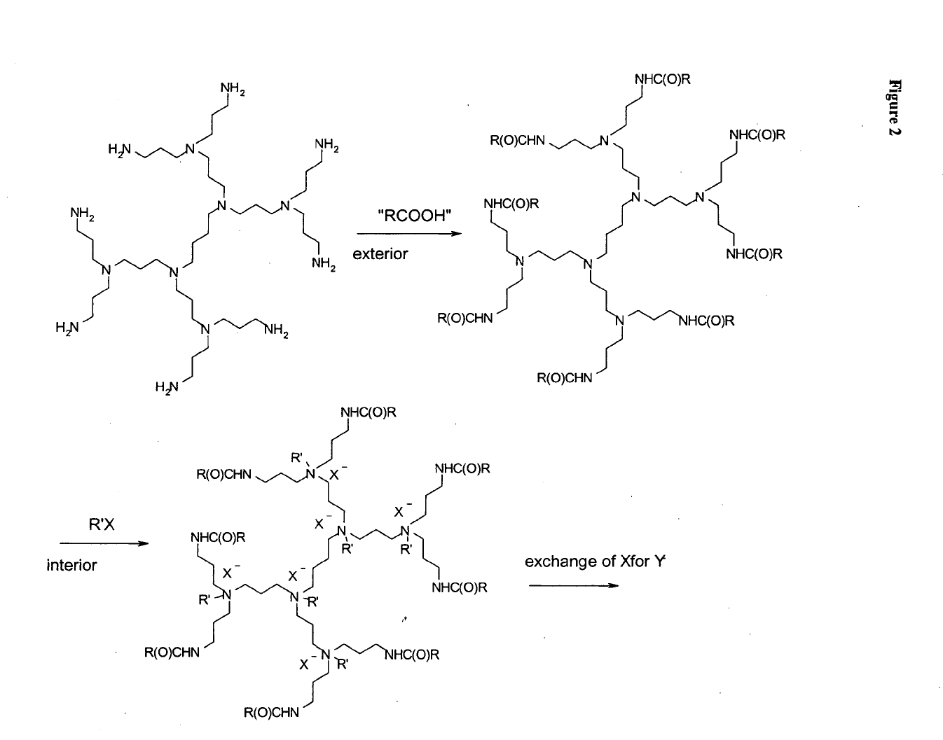

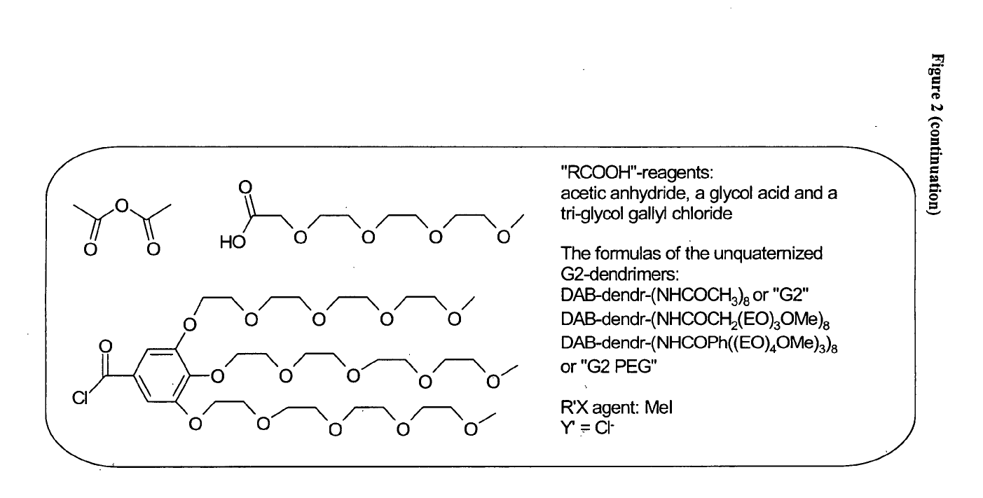

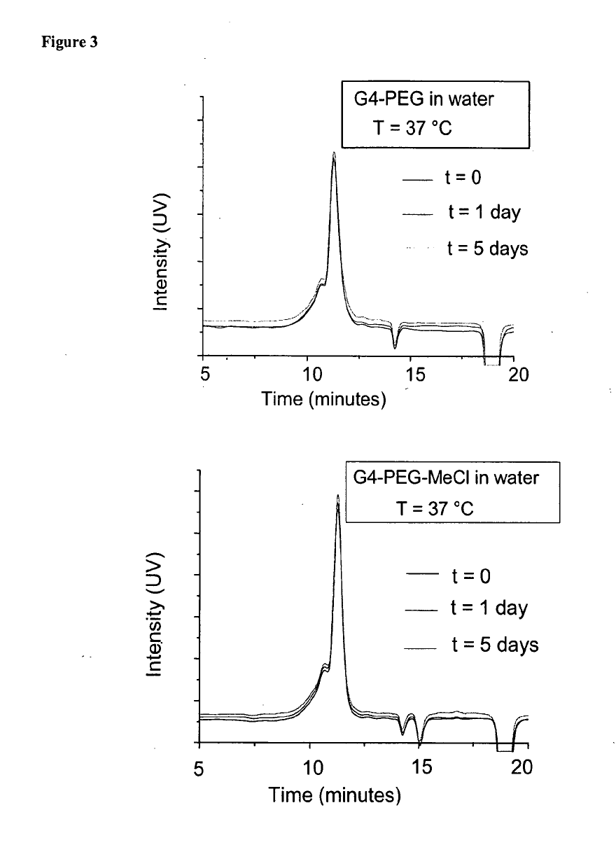

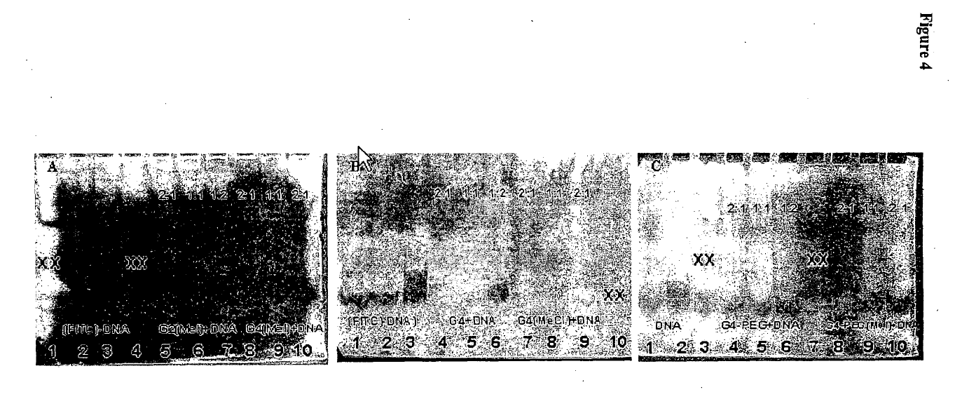

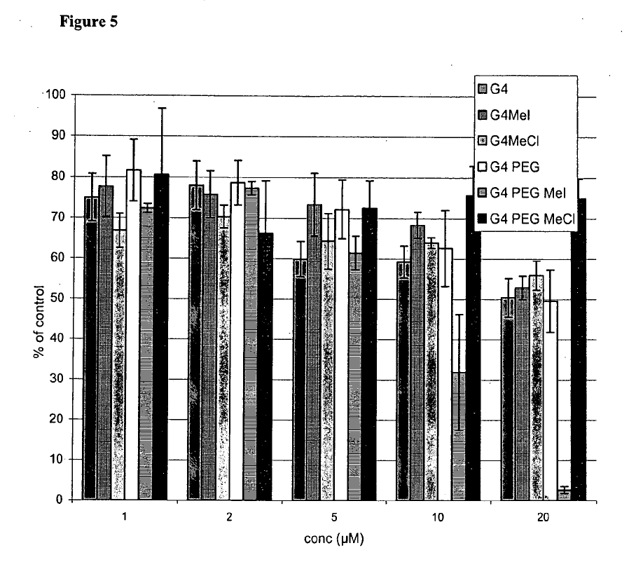

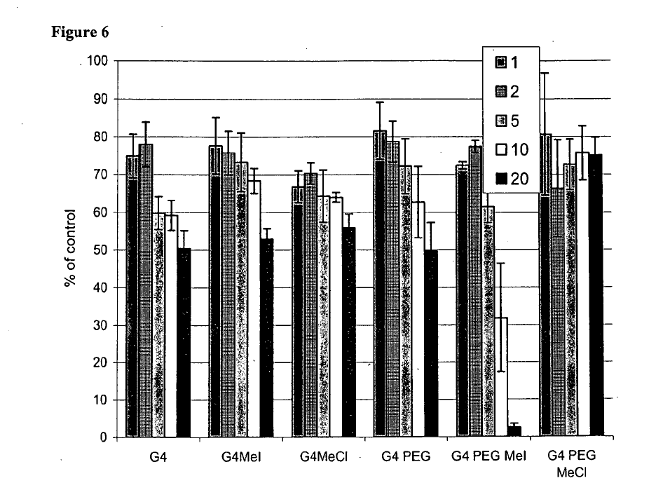

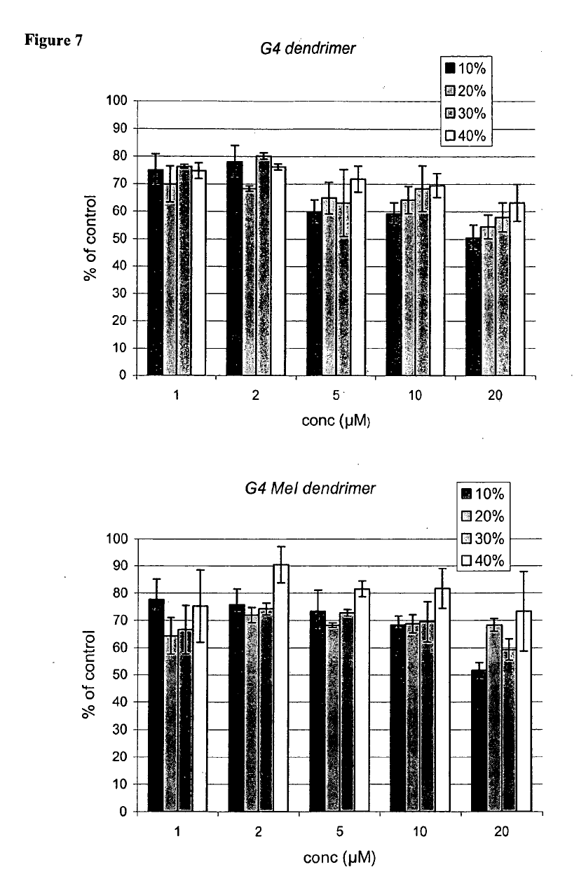

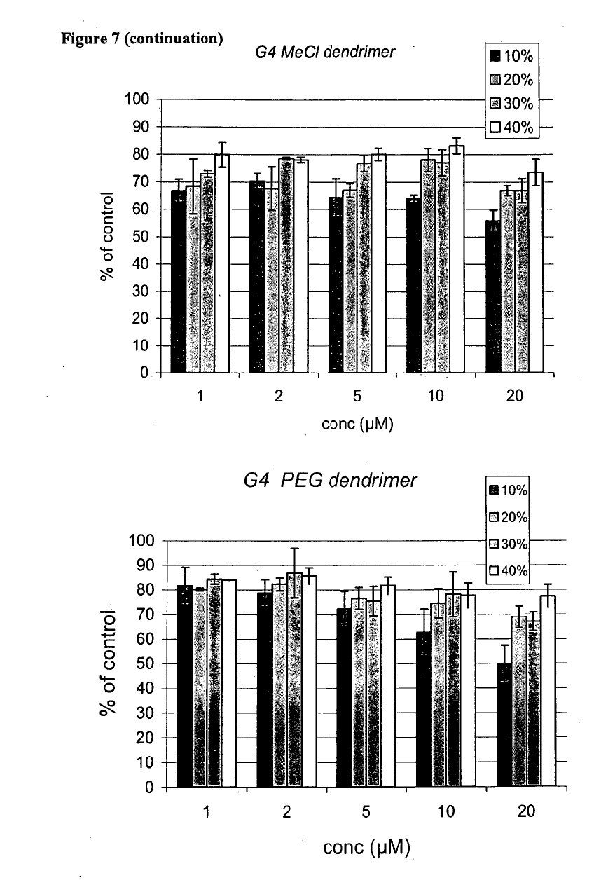

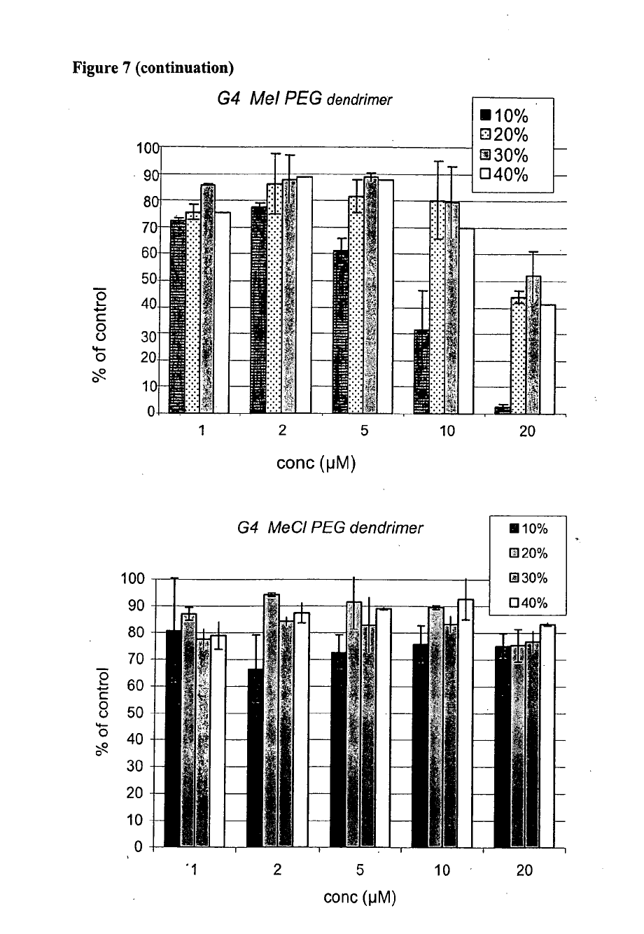

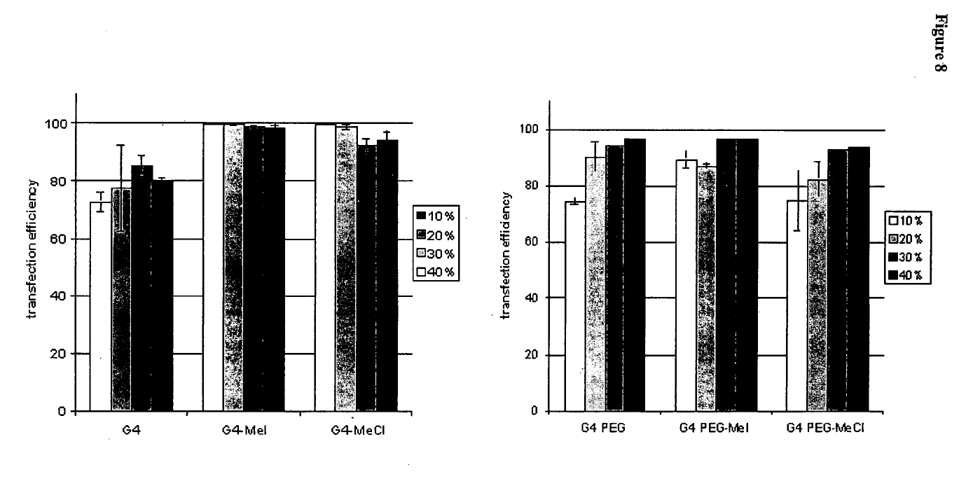

### LIST OF REFERENCES

1. Bakker, A., Floren, W., Voeten, J., Janssens, B., Smets, G., Wouters, W., and Janicot, M. (2001): Automation of whole body imaging of GFP-expressing tumors in living animals. G.I.T. Imaging & Microscopy, 03/2001:52-54.
2. Bielinska, A., Kukowska-Latallo, J. F., Johnson, J., Tomalia, D. A., and Baker, J. R., Jr. (1996): Regulation of in vitro gene expression using antisense oligonucleotides or antisense expression plasmids transfected using starburst PAMAM dendrimers. Nucleic Acids Res., 24:2176-2182.
3. Bielinska, A. U., Chen, C., Johnson, J., and Baker, J. R., Jr. (1999): DNA complexing with polyamidoamine dendrimers: implications for transfection. Bioconjug. Chem, 10:843-850.
4. Bosman, A. W., Janssen, H. M., and Meijer, E. W. (1999): About Dendrimers: Structure, Physical Properties, and Applications. Chem. Rev, 99:1665-1688.
5. Cheng, H., Zhou, R., Liu, L., Du, B., and Zhuo, R. (2000): Cyclic core dendrimer as a new kind of vector for gene transfer into mammalian cells. Genetica, 108:53-56.
6. Choi J, Joo D K, Kim C H, Kim K, and Park J S (2000) Synthesis of a Barbell-like Triblock Copolymer, Poly(L-lysine) Dendrimer-block-Poly(ethylene glycol)-block-Poly(L-lysine) Dendrimer and its self-assembly with plasmid DNA. J Am Chem Soc 122, 474-480.
7. de Brabander-van den Berg, E. M. M., Meijer. E. W. (1993) Poly(propylene imine) dendrimers: Large scale synthesis by heterogeneously catalyzed hydrogenations. Angew. Chem. Int. Ed. Engl., 32, 1308-1311.
8. Delong, R., Stephenson, K., Loftus, T., Fisher, M., Alahari, S., Nolting, A., and Juliano, R. L. (1997): Characterization of complexes of oligonucleotides with polyamidoamine starburst dendrimers and effects on intracellular delivery. J. Pharm. Sci, 86:762-764.
9. Duncan, R., Malik, N. (1996): Dendrimers: Biocompatibility and potential for delivery of anticancer agents. Proceed. Int. Symp. Control. Rel. Bioact. Mater., 23, 105-106.
10. Elissen-Roman, C., van Hest, J. C. M., Baars, M. W. P. L., van Genderen, M. H. P., Meijer, E. W. (1997) Amphiphilic block copolymers based on quaternized poly(propylene imine) dendrimers, PMSE, 77, 145-146.
11. Esfand, R. and Tomalia, D. A. (2001): Poly(amidoamine) (PAMAM) dendrimers: from biomimicry to drug delivery and biomedical applications. Drug Discov. Today, 6:427-436.
12. Haensler, J. and Szoka, F. C., Jr. (1993): Polyamidoamine cascade polymers mediate efficient transfection of cells in culture. Bioconjug. Chem, 4:372-379.
13. Hong, S., Bielinska, A. U., Mecke, A., Keszler, B., Beals, J. L., Shi, X., Balogh, L., Orr, B. G., Baker, J. R., Jr., and Banaszak Holl, M. M. (2004): Interaction of poly(amidoamine) dendrimers with supported lipid bilayers and cells: hole formation and the relation to transport. Bioconjug. Chem, 15:774-782.
14. Joester, D., Losson, M., Pugin, R., Heinzelmann, H., Walter, E., Merkle, H. P., and Diederich, F. (2003): Amphiphilic dendrimers: novel self-assembling vectors for efficient gene delivery. Angew Chem Int Ed Engl, 42:1486-1490.
15. Kabanov, V. A., Sergeyev, V. G., Pyshkina, O. A., Zinchenko, A. A., Zezin, A. B., Joosten, J. G. H., Brackman, J., Yoshikawa, K. (2000) Interpolyelectrolyte complexes formed by DNA and astramol poly(propylene imine) dendrimers, Macromolecules, 33, 9587-9593.
16. Kihara, F., Arima, H., Tsutsumi, T., Hirayama, F., and Uekama, K. (2003): In vitro and in vivo gene transfer by an optimized alpha-cyclodextrin conjugate with polyamidoamine dendrimer. Bioconjug. Chem, 14:342-350.
17. Kreider, J. L., Ford, W. T. (2001) Quaternary ammonium ion dendrimers from methylation of poly(propylene imine)s, J. Polym. Sci., Part A.: Polym. Chem., 39, 821-832.
18. Kukowska-Latallo, J. F., Bielinska, A. U., Johnson, J., Spindler, R., Tomalia, D. A., and Baker, J. R., Jr. (1996): Efficient transfer of genetic material into mammalian cells using Starburst polyamidoamine dendrimers. Proc. Natl. Acad. Sci U.S.A, 93:4897-4902.
19. Lee, J. H., Lim, Y. B., Choi, J. S., Lee, Y., Kim, T. I., Kim, H. J., Yoon, J. K., Kim, K., and Park, J. S. (2003): Polyplexes assembled with internally quaternized PAMAM-OH dendrimer and plasmid DNA have a neutral surface and gene delivery potency. Bioconjug. Chem, 14:1214-1221.
20. Liu M, Kono K, and Frechet, J. M. (1999) Water soluble dendrimer polyethylene glycol starlike conjugates as potential drug carriers. J Polym Sci Part A: Polym Chem 37, 3492-3503.
21. Liu, M. and Frechet, J. M. (1999): Designing dendrimers for drug delivery. Pharm. Sci. Technol. Today, 2:393-401.
22. Loup C, Zanta M A, Caminade A M, Majoral J P, and Meunier B. (1999) Preparation of Water-Soluble Cationic Phosphorus-Containing Dendrimers as DNA Transfecting Agents, Chem Eur J., 5, 3644-3650.
23. Luo D, Haverstick K, Belcheva N, Han E, and Saltzman W M. (2002) Polyethylene glycol conjugated PAMAM dendrimer for biocompatible, high efficiency DNA delivery. Macromolecules 35, 3456-3462.
24. Malik, N., Wiwattanapatapee, R., Klopsch, R., Lorenz, K., Frey, H., Weener, J. W., Meijer, E. W., Paulus, W., and Duncan, R. (2000): Dendrimers: relationship between structure and biocompatibility in vitro, and preliminary studies on the biodistribution of 125I-labelled polyamidoamine dendrimers in vivo. J Control Release, 65:133-148.
25. Ohsaki, M., Okuda, T., Wada, A., Hirayama, T., Niidome, T., and Aoyagi, H. (2002): In vitro gene transfection using dendritic poly(L-lysine). Bioconjug. Chem, 13:510-517.
26. Ottaviani M F, Furini F, Casini A, Turro N J, Jockusch S, Tomalia D A, and Messori L. (2000) Formation of supramolecular structures between DNA and starburst dendrimers studied by EPR, CD, UV and melting profiles. Macromolecules 33, 7842-7851.
27. Pan, Y., Ford, W. T. (1999) Dendrimers with both hydrophilic and hydrophobic chains at every end, Macromolecules, 32, 5468-5470.
28. Pan, Y., Ford, W. T. (2000) Amphiphilic dendrimers with both octyl and triethylenoxy methyl ether chain ends, Macromolecules, 33, 3731-3738.
29. Patri, A. K., Majoros, I. J., and Baker, J. R. (2002): Dendritic polymer macromolecular carriers for drug delivery. Curr. Opin. Chem. Biol., 6:466-471.
30. Qin, L., Pahud, D. R., Ding, Y., Bielinska, A. U., Kukowska-Latallo, J. F., Baker, J. R., Jr., and Bromberg, J. S. (1998): Efficient transfer of genes into murine cardiac grafts by Starburst polyamidoamine dendrimers. Hum. Gene Ther., 9:553-560.
31. Roberts, J. C., Bhalgat, M. K., and Zera, R. T. (1996): Preliminary biological evaluation of polyamidoamine (PAMAM) Starburst dendrimers. J. Biomed. Mater. Res., 30:53-65.
32. Schluter D A. Synthesis of Polymers, Materials Science and Technology Series. Janssen, H. M. and Meijer E W. Chapter 12: The synthesis and Characterization of Dendritic Molecules. 1999. Wiley-VCH Verlag GmbH D-69469 Weinheim, Germany.
33. Shah, D. S., Sakthivel, T., Toth, I., Florence, A. T., and Wilderspin, A. F. (2000): DNA transfection and transfected cell viability using amphipathic asymmetric dendrimers. Int J. Pharm., 208:41-48.
34. Shchepinov, M. S., Udalova, I. A., Bridgman, A. J., and Southern, E. M. (1997): Oligonucleotide dendrimers: synthesis and use as polylabelled DNA probes. Nucleic Acids Res., 25:4447-4454.
35. Stiriba S. E., Frey H, and Haag R. (2002) Dendritic Polymers in Biomedical Applications: From Potential to clinical Use in Diagnostics and Therapy. Angew Chem Int Ed 41, 1329-1334.
36. Tang, M. X., Redemann, C. T., and Szoka, F. C., Jr. (1996): In vitro gene delivery by degraded polyamidoamine dendrimers. Bioconjug. Chem, 7:703-714.
37. Tang, M. X. and Szoka, F. C. (1997): The influence of polymer structure on the interactions of cationic polymers with DNA and morphology of the resulting complexes. Gene Ther., 4:823-832.
38. Thomas, T. P., Patri, A. K., Myc, A., Myaing, M. T., Ye, J. Y., Norris, T. B., and Baker, J. R., Jr. (2004): In vitro targeting of synthesized antibody-conjugated dendrimer nanoparticles. Biomacromolecules., 5:2269-2274.
39. Wörner, C., Mülhaupt, R. (1993), Polynitrile- and polyamine-functional poly(trimethylene imine) dendrimers, Angew. Chem. Int. Ed. Engl., 32, 1306-1308.
40. Wu, J., Zhou, J., Qu, F., Bao, P., Zhang, Y., and Peng, L. (2005): Polycationic dendrimers interact with RNA molecules: polyamine dendrimers inhibit the catalytic activity of Candida ribozymes. Chem. Commun. (Camb.), 313-315.
41. Yoo, H., Sazani, P., and Juliano, R. L. (1999): PAMAM dendrimers as delivery agents for antisense oligonucleotides. Pharm. Res., 16:1799-1804.
42. Zinselmeyer, B. H., Mackay, S. P., Schatzlein, A. G., and Uchegbu, I. F. (2002): The lower-generation polypropylenimine dendrimers are effective gene-transfer agents. Pharm. Res., 19:960-967.

1. Modified poly-(propylene imine) dendrimer of generation 1, 2, 3, 4 or 5, also comprising incomplete dendrimers and mixtures thereof, comprising external end groups and internal amine groups, characterized in that:
(a) substantially all external end groups are groups of formula (I) 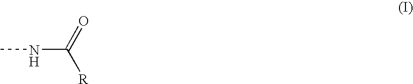
wherein R is a radical selected from the group of C₁₋₁₀alkyl, polyethylene glycol radical of formula 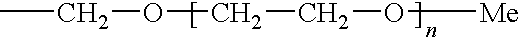
wherein n is 3, 4, 5, 6, 7, 8, 9, 10, 11, or 12; and
polyethylene glycol gallyl radical of formula 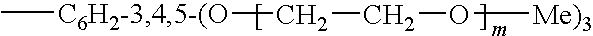
wherein each m independently is 1, 2, 3, 4, 5, 6, 7, 8, 9, 10, 11, or 12; and
(b) substantially all internal amine groups are cationic quaternary ammonium groups.

2. Modified poly-(propylene imine) dendrimer of generation 1, 2, 3, 4 or 5, also comprising incomplete dendrimers and mixtures thereof, characterized in that the modified poly-(propylene imine) dendrimer is obtained by:
(a) first reacting a poly-(propylene imine) dendrimer substantially comprising external amine end groups and internal tertiary amine groups, with an acylation agent selected from the group of acetic anhydride, a C₁₋₁₀alkyl halide, a polyethylene glycol acid of formula 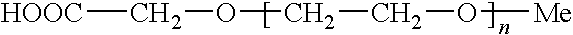
wherein n is 3, 4, 5, 6, 7, 8, 9, 10, 11, or 12; and
polyethylene glycol gallyl halide of formula 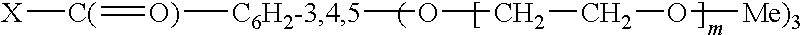
wherein each m independently is 1, 2, 3, 4, 5, 6, 7, 8, 9, 10, 11, or 12 and X is halide; and
(b) reacting the product obtained in step (a) with a quaternization agent.

3. Modified poly-(propylene imine) dendrimer according to claim 2, characterized in that the halide is a chloride.

4. Modified poly-(propylene imine) dendrimer according to claim 1, characterized in that the dendrimer is selected from the group of quaternized
DAB-dendr-(NHCOCH₃)₄,
DAB-dendr-(NHCOCH₃)₈,
DAB-dendr-(NHCOCH₃)₁₆,
DAB-dendr-(NHCOCH₃)₃₂,
DAB-dendr-(NHCOCH₃)₆₄,
DAB-dendr-(NHCOPh((EO)₄OMe)₃)₄,
DAB-dendr-(NHCOPh((EO)₄OMe)₃)₈,
DAB-dendr-(NHCOPh((EO)₄OMe)₃)₁₆,
DAB-dendr-(NHCOPh((EO)₄OMe)₃)₃₂ and
DAB-dendr-(NHCOPh((EO)₄OMe)₃)₆₄.

5. Pharmaceutical composition, suitable for administration to a mammal, characterized in that it comprises: (a) the modified poly-(propylene imine) dendrimer according to claim 1; and (b) an anionic bioactive therapeutic factor.

6. Pharmaceutical composition according to claim 5, characterized in that the anionic bioactive therapeutic factor is selected from the group of pharmaceutical active compounds, nucleic acids, nucleic acid sequences, oligomers of DNA and RNA, polynucleotides, DNAzymes, single and double stranded DNA, single and double stranded RNA, antisense RNA and DNA, hammerhead RNA, short interfering RNA, micro RNA, ribozymes and the like; or combinations thereof.

7. Pharmaceutical composition according to claim 6, characterized in that the anionic bioactive therapeutic factor has a molecular weight of equal to or less than 5,000 dalton.

8. Use of a compound according to claim 1 as a transfection agent for an anionic bioactive therapeutic factor.

9. Use of a pharmaceutical composition according to claim 5 in gene therapy.

10. Use of a pharmaceutical composition according to claim 5 for the treatment of a cancer tumor, associated with the liver, kidney, acute lymphoblastic leukemia, acute myeloid leukemia, Ewing's sarcoma, gestational trophoblastic carcinoma, Hodgkin's disease, non-Hodgkin's lymphoma, Burkitt's lymphoma diffuse large cell lymphoma, follicular mixed lymphoma, lymphoblastic lymphoma, rhabdomyosarcoma, testicular carcinoma, Wilms's tumor, anal carcinoma, bladder carcinoma breast carcinoma, chronic lymphocytic leukemia, chronic myelogenous leukemia, hairy cell leukemia, head and neck carcinoma, lung (small cell) carcinoma, multiple myeloma, follicular lymphoma, ovarian carcinoma, brain tumors (astrocytoma), cervical carcinoma, colorectal carcinoma, hepatocellular carcinoma, Karposi's sarcoma, lung (non-small-cell) carcinoma, melanoma, pancreatic carcinoma, prostate carcinoma, soft tissue sarcoma, breast carcinoma, colorectal carcinoma (stage III), osteogenic sarcoma, ovarian carcinoma (stage III), or combinations thereof.

11. Modified poly-(propylene imine) dendrimer of generation 1, 2, 3, 4 or 5, also comprising incomplete dendrimers and mixtures thereof, comprising external end groups and internal amine groups, characterized in that:
substantially all external end groups are groups of formula (I), wherein R is a radical selected from the group of C₁₋₁₀alkyl, polyethylene glycol radical of formula 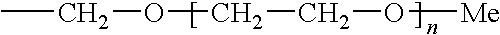
wherein n is 3, 4, 5, 6, 7, 8, 9, 10, 11, or 12; and
polyethylene glycol gallyl radical of formula 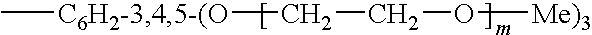
wherein each m independently is 1, 2, 3, 4, 5, 6, 7, 8, 9, 10, 11, or 12.

12. Modified poly-(propylene imine) dendrimer of generation 1, 2, 3, 4 or 5, also comprising incomplete dendrimers and mixtures thereof, characterized in that the modified poly-(propylene imine) dendrimer is obtained by:
first reacting a poly-(propylene imine) dendrimer substantially comprising external amine end groups and internal tertiary amine groups, with an acylation agent selected from the group of acetic anhydride, a C₁₋₁₀alkyl halide, a polyethylene glycol acid of formula 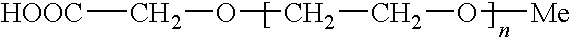
wherein n is 3, 4, 5, 6, 7, 8, 9, 10, 11, or 12; and
polyethylene glycol gallyl halide of formula 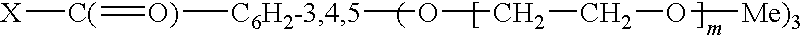
wherein each m independently is 1, 2, 3, 4, 5, 6, 7, 8, 9, 10, 11, or 12 and X is halide.


SEQUENCE LISTING (1 sequence)

SEQ ID NO: 1 - DNA, 32 bp, Artificial Sequence
  Description of Artificial Sequence Synthetic oligonucleotide
tgaggggcaggctagctacaacgacgtcgcgg